(19) United States
(12) Patent Application Publication
(10) Pub. No.: US 2024/0138712 A1
(43) Pub. Date: May 2, 2024

(54) FEATURE-AMOUNT GENERATION DEVICE, GAIT MEASUREMENT SYSTEM, FEATURE-AMOUNT GENERATION METHOD, AND RECORDING MEDIUM

(71) Applicant: NEC Corporation, Tokyo (JP)

(72) Inventors: Chenhui HUANG, Tokyo (JP); Fumiyuki NIHEY, Tokyo (JP); Kentaro NAKAGARA, Tokyo (JP); Kenichiro FUKUSHI, Tokyo (JP)

(73) Assignee: NEC Corporation, Tokyo (JP)

(21) Appl. No.: 18/393,969

(22) Filed: Dec. 22, 2023

Related U.S. Application Data
(63) Continuation of application No. 18/266,414, filed on Jun. 9, 2023.

(51) Int. Cl. A61B 5/11; A61B 5/00

(52) U.S. Cl. CPC A61B 5/112; A61B 5/7235

(57) ABSTRACT

A feature-amount generation device that includes an extraction unit that generates a gait waveform for one gait cycle from time-series data of sensor data related to a motion of a foot, extracts a feature amount from the generated gait waveform, and extracts a gait phase cluster by integrating a plurality of temporally continuous gait phases from each of which a feature amount is extracted, and a generation unit that generates a feature amount of the gait phase cluster using a preset feature-amount constitutive expression, and generates feature-amount data in which the plurality of gait phases constituting the gait phase cluster and the feature amount of the gait phase cluster are associated with each other.

This application is a continuation application of U.S. patent application Ser. No. 18/266,414 filed on Jun. 9, 2023, which is a National Stage Entry of PCT/JP2021/012132 filed on Mar. 24, 2021, the contents of all of which are incorporated herein by reference, in their entirety.

### TECHNICAL FIELD

The present disclosure relates to a feature-amount generation device or the like that generates a feature amount from data related to a gait.

### BACKGROUND ART

With increasing interest in healthcare that performs physical condition management, a service in which a gait including a gait feature is measured and that provides information related to the gait to a user has attracted attention. The physical feature affects a gait. Therefore, the feature appearing in the gait can be used to estimate the physical feature. For example, the physical feature of the user can be estimated by measuring sensor data related to a gait by using an inertial measurement device mounted on footwear and verifying a feature appearing in the gait of the user based on the measured sensor data.

PTL 1 discloses a gait analysis system that analyzes a gait state of a pedestrian. The system of PTL 1 includes a gait sensor, a wireless communication device, and a gait analysis device. The gait sensor is attached to a foot of a pedestrian who performs gait training rehabilitation. The gait sensor wirelessly outputs detection data of acceleration or angular velocity. The gait analysis device acquires detection data via the wireless communication device. The gait analysis device calculates a rehabilitation analysis item related to the foot portion based on the acquired detection data.

PTL 2 discloses an insurance proposal system that proposes an insurance plan based on gait information about a user. The system of PTL 2 includes a gait information acquisition unit, a pedestrian database, and an index value calculation unit. The gait information acquisition unit acquires the gait information about the user from the footwear module including the sensor unit that detects the motion. In the pedestrian database, gait information about a plurality of pedestrians and injuries and sickness history information indicating a past injuries and sickness stored in association with the gait information are accumulated as pedestrian information. The index value calculation unit refers to the gait information and the pedestrian information about the user, and calculates an index value serving as an index of an insurance premium for the user.

PTL 3 discloses an information processing device that extracts a feature amount from motion information about a foot. The device of PTL 3 includes an acquisition unit and a feature-amount extraction unit, and the acquisition unit acquires motion information about the foot measured by a motion measurement device provided on the foot of the user. The feature-amount extraction unit extracts a feature amount used for user identification from time-series data of at least one gait cycle included in the motion information.

### CITATION LIST

### Patent Literature

PTL 1: JP 2008-161228 A
PTL 2: JP 2020-197948 A
PTL 3: WO 2020/240751 A1

### SUMMARY OF INVENTION

### Technical Problem

A wide variety of features are appearing in the gait. Therefore, in order to construct a model that can be used to estimate a physical feature, it is necessary to collect a lot of data in order to verify a wide variety of features appearing in a gait.

In the method of PTL 1, a rehabilitation analysis item is calculated by using a velocity or an angle calculated by integrating acceleration or angular velocity detection data. In the method of PTL 1, detection data for calculating a rehabilitation analysis item is transmitted from a gait sensor to a gait analysis device. Therefore, in the method of PTL 1, the amount of data communication increases in order to calculate rehabilitation analysis item.

In the method of PTL 2, an index value serving as an index of an insurance premium is calculated using gait information based on sensing data detected by a sensor unit. In the method of PTL 2, sensing data for calculating an index value is transmitted from a sensor unit to a gait information acquisition unit. Therefore, in the method of PTL 2, the amount of data communication between the sensor unit and the gait information acquisition unit increases in order to calculate an accurate index value.

In the method of PTL 3, a feature amount is extracted from time-series data of at least one gait cycle. In the method of PTL 3, the motion information from which the feature amount is extracted is transmitted from the motion measurement device provided on the foot of the user to the information processing device. That is, in the method of PTL 3, it is necessary to transmit the motion information for one gait cycle from the motion measurement device to the information processing device in order to identify the user.

An object of the present disclosure is to provide a feature-amount generation device and the like capable of reducing a data amount for verifying a feature appearing in a gait.

### Solution to Problem

A feature-amount generation device according to an aspect of the present disclosure includes an extraction unit that generates a gait waveform for one gait cycle from time-series data of sensor data related to a motion of a foot, extracts a feature amount from the generated gait waveform, and extracts a gait phase cluster by integrating a plurality of temporally continuous gait phases from each of which a feature amount is extracted, and a generation unit that generates a feature amount of the gait phase cluster using a preset feature-amount constitutive expression, and generates feature-amount data in which the plurality of gait phases constituting the gait phase cluster and the feature amount of the gait phase cluster are associated with each other.

A feature-amount generation method according to an aspect of the present disclosure includes generating a gait waveform for one gait cycle from time-series data of sensor data related to a motion of a foot, extracting a feature amount from the generated gait waveform, extracting a gait phase cluster by integrating a plurality of temporally continuous gait phases from each of which a feature amount is extracted, generating a feature amount of the gait phase cluster using a feature-amount constitutive expression, and generating feature-amount data in which the plurality of gait phases constituting the gait phase cluster and the feature amount of the gait phase cluster are associated with each other.

A program according to an aspect of the present disclosure causes a computer to execute a process of generating a gait waveform for one gait cycle from time-series data of sensor data related to a motion of a foot, a process of extracting a feature amount from the generated gait waveform, a process of extracting a gait phase cluster by integrating a plurality of temporally continuous gait phases from each of which a feature amount is extracted, a process of generating a feature amount of the gait phase cluster using a feature-amount constitutive expression, and a process of generating feature-amount data in which the plurality of gait phases constituting the gait phase cluster and the feature amount of the gait phase cluster are associated with each other.

### Advantageous Effects of Invention

According to the present disclosure, it is possible to provide a feature-amount generation device and the like capable of reducing a data amount for verifying a feature appearing in a gait.

### BRIEF DESCRIPTION OF DRAWINGS

FIG. 1 is a block diagram illustrating an example of a configuration of a gait measurement system according to a first example embodiment.

FIG. 2 is a conceptual diagram illustrating an arrangement example of a measurement device of the gait measurement system according to the first example embodiment.

FIG. 3 is a conceptual diagram for describing a coordinate system set in the measurement device of the gait measurement system according to the first example embodiment.

FIG. 4 is a conceptual diagram for describing a human body surface applied to the measurement device of the gait measurement system according to the first example embodiment.

FIG. 5 is a conceptual diagram for explaining a gait cycle.

FIG. 6 is a block diagram illustrating an example of a configuration of the measurement device of the gait measurement system according to the first example embodiment.

FIG. 7 is a conceptual diagram for describing an example of extraction of a gait phase cluster by the measurement device of the gait measurement system according to the first example embodiment.

FIG. 8 is a conceptual diagram for describing generation of a feature amount of a gait phase cluster by the measurement device of the gait measurement system according to the first example embodiment.

FIG. 9 is a block diagram illustrating an example of a configuration of a data processing device of the gait measurement system according to the first example embodiment.

FIG. 10 is a flowchart for explaining an example of an operation of the measurement device of the gait measurement system according to the first example embodiment.

FIG. 11 is a block diagram illustrating an example of a configuration of a gait measurement system according to a second example embodiment.

FIG. 12 is a block diagram illustrating an example of a configuration of a measurement device of the gait measurement system according to the second example embodiment.

FIG. 13 is a block diagram illustrating an example of a configuration of a data processing device of the gait measurement system according to the second example embodiment.

FIG. 14 is a block diagram illustrating an example of a configuration of a training system according to a third example embodiment.

FIG. 15 is a block diagram illustrating an example of a configuration of a training device of a training system according to the third example embodiment.

FIG. 16 is a conceptual diagram for describing an example of training with feature-amount data in a training system according to the third example embodiment.

FIG. 17 is a block diagram illustrating an example of a configuration of a gait measurement system according to a fourth example embodiment.

FIG. 18 is a block diagram illustrating an example of a configuration of a data processing device of the gait measurement system according to the fourth example embodiment.

FIG. 19 is a conceptual diagram for describing an example of estimation in the gait measurement system according to the fourth example embodiment.

FIG. 20 is a conceptual diagram illustrating a display example of an estimation result by the gait measurement system according to the fourth example embodiment.

FIG. 21 is a block diagram illustrating an example of a configuration of the gait measurement system according to a fifth example embodiment.

FIG. 22 is a graph for explaining a verification example of a gait phase cluster extracted from a gait waveform of one gait cycle by the measurement device of each example embodiment.

FIG. 23 is a graph for explaining a verification example of a gait phase cluster extracted from a gait waveform of one gait cycle by the measurement device of each example embodiment.

FIG. 24 is a graph for explaining a verification example of a gait phase cluster extracted from a gait waveform of one gait cycle by the measurement device of each example embodiment.

FIG. 25 is a graph for explaining a verification example of a gait phase cluster extracted from a gait waveform of one gait cycle by the measurement device of each example embodiment.

FIG. 26 is a graph for explaining a verification example of a gait phase cluster extracted from a gait waveform of one gait cycle by the measurement device of each example embodiment.

FIG. 27 is a graph for explaining a verification example of a gait phase cluster extracted from a gait waveform of one gait cycle by the measurement device of each example embodiment.

FIG. 28 is a correspondence table summarizing gait motions related to gait phase clusters extracted from a gait waveform of one gait cycle by the measurement device of each example embodiment.

FIG. 29 is a graph in which an estimation value of a first metatarsophalangeal angle estimated using an estimation model trained by linear regression is associated with a true value of the first metatarsophalangeal angle.

FIG. 30 is a table summarizing parameters related to a correlation between an estimation value of the first metatarsophalangeal angle estimated using an estimation model trained using various algorithms and a true value of the first metatarsophalangeal angle.

FIG. 31 is a block diagram illustrating an example of a hardware configuration that implements control and processing of each example embodiment.

### EXAMPLE EMBODIMENT

Hereinafter, example embodiments of the present invention will be described with reference to the drawings. However, the example embodiments described below have technically preferable limitations for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, a gait measurement system according to the first example embodiment will be described with reference to the drawings. The gait measurement system of the present example embodiment measures sensor data related to a physical quantity related to a motion of a foot by a sensor installed at footwear worn by a user. For example, the physical quantity related to a motion of the foot is acceleration in three axial directions (also referred to as spatial acceleration) measured by the acceleration sensor, angular velocities around the three axes (also referred to as spatial angular velocities) measured by the angular velocity sensor, and the like. The gait measurement system of the present example embodiment extracts a feature amount related to the gait from time-series data (also referred to as a gait waveform) of the measured sensor data. The gait measurement system of the present example embodiment extracts a feature amount by the sensor to transmit the extracted feature amount from the sensor to the data processing unit.

(Configuration)

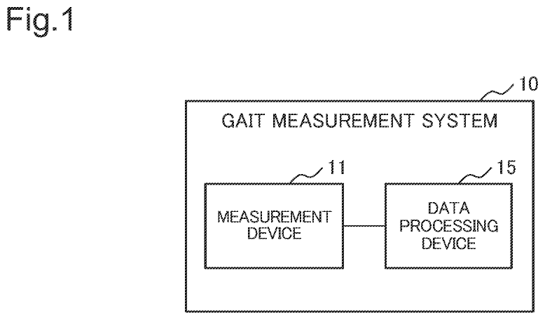

FIG. 1 is a block diagram illustrating an example of a configuration of a gait measurement system 10 of the present example embodiment. The gait measurement system 10 includes a measurement device 11 and a data processing device 15. The measurement device 11 and the data processing device 15 may be connected by wire or wirelessly. The measurement device 11 and the data processing device 15 may be configured as a single device. Although only one measurement device 11 is illustrated in FIG. 1, one measurement device 11 (two in total) may be disposed at each of the left and right feet.

The measurement device 11 is installed at at least one of the left and right feet. For example, the measurement device 11 is installed at footwear such as shoes. In the present example embodiment, an example will be described in which the measurement devices 11 are disposed at a position on the back side of the arch of each of the left and right feet. The measurement device 11 includes an acceleration sensor and an angular velocity sensor. The measurement device 11 measures, as a physical quantity related to a motion of the foot of the user wearing the footwear, a physical quantity related to a motion of the foot such as acceleration in three axial directions (also referred to as spatial acceleration) and angular velocities around the three axes (also referred to as spatial angular velocities). The physical quantity related to a motion of the foot measured by the measurement device 11 includes not only the acceleration and the angular velocity but also the velocity and the angle calculated by integrating the acceleration and the angular velocity. The physical quantity related to a motion of the foot measured by the measurement device 11 also includes a position (trajectory) calculated by second-order integration of the acceleration.

For example, the measurement device 11 is disposed at an insole inserted into the shoe 100. For example, the measurement device 11 is disposed at the bottom face of the shoe 100. For example, the measurement device 11 is embedded in the main body of the shoe 100. The measurement device 11 may be detachable from the shoe 100 or may not be detachable from the shoe 100. The measurement device 11 may be disposed at a position other than that of the back side of the arch of foot as long as sensor data related to a motion of the foot can be acquired. The measurement device 11 may be installed at a decorative article such as a sock, a supporter, or an anklet. The measurement device 11 may be directly attached to the foot or may be embedded in the foot.

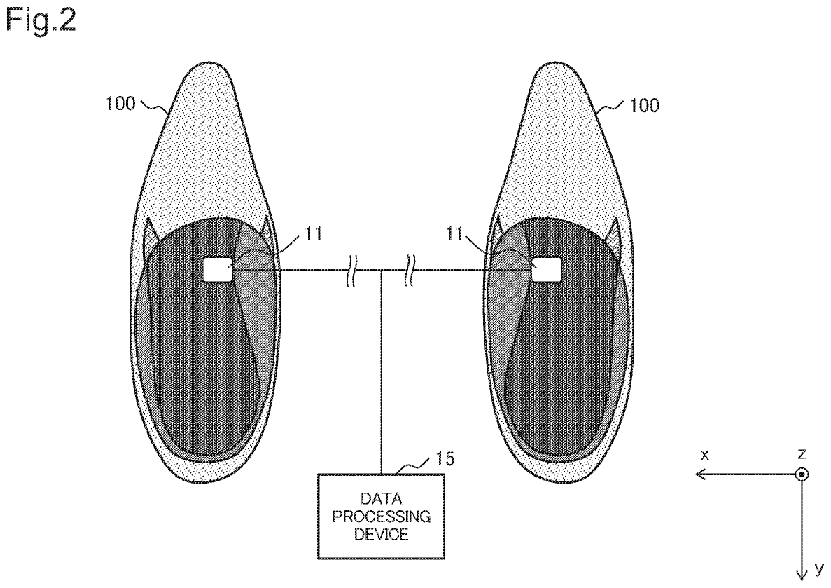

FIG. 2 is a conceptual diagram illustrating an example in which the measurement device 11 is disposed at the shoe 100. In the example of FIG. 2, the measurement device 11 is disposed at a position related to the back side of the arch of each of the left and right feet. In the example of FIG. 2, the measurement devices 11 produced with the same specification without distinction between the left and right are disposed at the left and right shoes 100. It is assumed that the vertical directions (directions in the Z-axis direction) of the measurement devices 11 disposed at the left and right shoes 100 are the same. That is, the axes set in the sensor data derived from the left foot and the sensor data derived from the right foot are common to the left and right. In the example of FIG. 2, a local coordinate system including an x-axis in the left-right direction, a y-axis in the front-rear direction, and a z-axis in the up-down direction is set with reference to the measurement device 11. In the x-axis, the left side is positive, in the y-axis, the rear side is positive, and in the z-axis, the upper side is positive. The direction of the axis set in the measurement device 11 is not limited to the example of FIG. 2 as long as the right and left feet are the same.

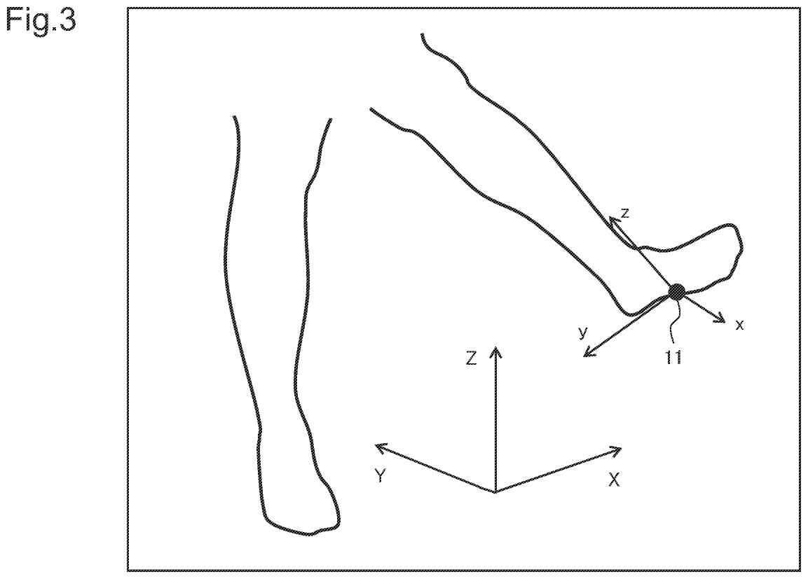

FIG. 3 is a conceptual diagram for explaining a local coordinate system (x-axis, y-axis, z-axis) set in the measurement device 11 and a world coordinate system (X-axis, Y-axis, Z-axis) set with respect to the ground in a case where the measurement device 11 is installed at a position corresponding to the back side of the arch of the foot. In the world coordinate system (X-axis, Y-axis, Z-axis), in a state where the user is standing upright, a lateral direction of the user is set to an X-axis direction (leftward direction is positive), a back face direction of the user is set to a Y-axis direction (rearward direction is positive), and a gravity direction is set to a Z-axis direction (vertically upward direction is positive).

The measurement device 11 is achieved by, for example, an inertial measurement device including an acceleration sensor and an angular velocity sensor. An example of the inertial measurement device is an inertial measurement unit (IMU). The IMU includes a three-axis acceleration sensor and a three-axis angular velocity sensor. Examples of the inertial measurement device include a vertical gyro (VG), an attitude heading (AHRS), and a global positioning system/inertial navigation system (GPS/INS).

The measurement device 11 converts the measured physical quantity into digital data (also referred to as sensor data). The measurement device 11 generates a waveform (also referred to as a gait waveform) for one gait cycle from the time-series data of the sensor data. For example, the measurement device 11 generates a gait waveform related to acceleration in three axial directions and angular velocities around the three axes. For example, the measurement device 11 generates a gait waveform related to the plantar angle using the acceleration in three axial directions and the angular velocities around the three axes. The gait waveform for one gait cycle is a set of sensor data for one gait cycle such as acceleration, an angular velocity, and a plantar angle. Hereinafter, a unit section of one gait cycle is referred to as a “gait phase”. For example, when the gait waveform for one gait cycle is equally divided into 100 in sections of 0 to 100% (%), the gait phase is 1%. The division reference of the gait waveform for one gait cycle is not particularly limited. For example, the gait waveform for one gait cycle may be divided according to the measurement condition of the data by the measurement device 11 or the gait to be measured.

The measurement device 11 extracts a feature amount from a gait waveform for one gait cycle. The feature amount extracted from each gait phase of the gait waveform is also referred to as a first feature amount. For example, the measurement device 11 extracts a feature amount related to the physical feature of the user from the gait waveform for one gait cycle. In the present example embodiment, attention is paid to the fact that the physical feature of the user affects the gait. The physical feature of the user affect acceleration, an angular velocity, a plantar angle, and the like. For example, when the user has a physical feature such as hallux valgus, a specific feature appears in the gait waveform of acceleration, an angular velocity, and a plantar angle.

When the feature amount is extracted from each of the temporally continuous gait phases, the measurement device 11 extracts a cluster (also referred to as a gait phase cluster) by integrating the gait phases. For example, the measurement device 11 may be configured to extract a feature amount from each gait phase constituting a gait phase cluster related to the physical feature of the measurement target based on a preset condition.

The measurement device 11 generates the feature amount of the gait phase cluster using the feature-amount constitutive expression. The feature amount of the gait phase cluster generated by the measurement device 11 using the feature-amount constitutive expression is also referred to as a second feature amount. The feature-amount constitutive expression is a preset calculation expression for generating the feature amount of the gait phase cluster. For example, the feature-amount constitutive expression is a calculation expression related to four arithmetic operations. For example, the feature amount (second feature amount) calculated using the feature-amount constitutive expression is an integral average value, an arithmetic average value, an inclination, a variation, or the like of the feature amount (first feature amount) in each gait phase included in the gait phase cluster. The measurement device 11 generates data (also referred to as feature-amount data) in which gait phases (gait cycle) constituting a gait phase cluster and a feature amount (second feature amount) of the gait phase cluster are associated with each other. In a case where the feature amount (first feature amount) is extracted from the single gait phase, the measurement device 11 generates feature-amount data in which the extracted feature amount is associated with the gait phase.

The measurement device 11 transmits the generated feature-amount data of the gait phase cluster to the data processing device 15. For example, the measurement device 11 is connected to the data processing device 15 constructed in the cloud via a mobile terminal (not illustrated) carried by the user. The mobile terminal (not illustrated) is a portable communication device. For example, the mobile terminal is a portable communication device having a communication function, such as a smartphone, a smart watch, or a mobile telephone. The mobile terminal receives, from the measurement device 11, the feature-amount data generated from the sensor data related to a motion of the foot of the user. The mobile terminal transmits the received feature-amount data to a server or the like on which the data processing device 15 is mounted. The function of the data processing device 15 may be achieved by an application installed in a mobile terminal. In this case, the mobile terminal processes the received feature amount by application software or the like installed in the mobile terminal itself.

The data processing device 15 receives the feature-amount data from the measurement device 11. The data processing device 15 performs data processing according to the setting using the received feature-amount data. The data processing device 15 may perform any data processing as long as the feature-amount data acquired from the measurement device 11 is used. For example, the data processing device 15 estimates a physical feature using the feature-amount data. The data processing device 15 outputs a result of the data processing. For example, the data processing device 15 outputs a result of the data processing to a display device (not illustrated) or an external system.

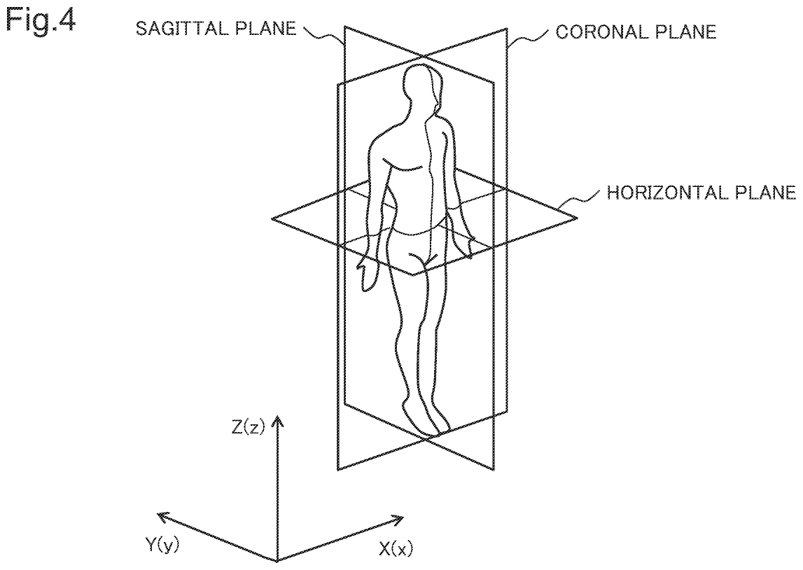

FIG. 4 is a conceptual diagram for explaining a face (also referred to as a human body surface) set for the human body. In the present example embodiment, a sagittal plane dividing the body into left and right, a coronal plane dividing the body into front and rear, and a horizontal plane dividing the body horizontally are defined. In the upright state as illustrated in FIG. 4, the world coordinate system coincides with the local coordinate system. In the present example embodiment, rotation in the sagittal plane with the x-axis as a rotation axis is defined as roll, rotation in the coronal plane with the y-axis as a rotation axis is defined as pitch, and rotation in the horizontal plane with the z-axis as a rotation axis is defined as yaw. A rotation angle in a sagittal plane with the x-axis as a rotation axis is defined as a roll angle, a rotation angle in a coronal plane with the y-axis as a rotation axis is defined as a pitch angle, and a rotation angle in a horizontal plane with the z-axis as a rotation axis is defined as a yaw angle. In the present example embodiment, when the body is viewed from behind, counterclockwise rotation in the coronal plane is defined as positive, and clockwise rotation in the coronal plane is defined as negative.

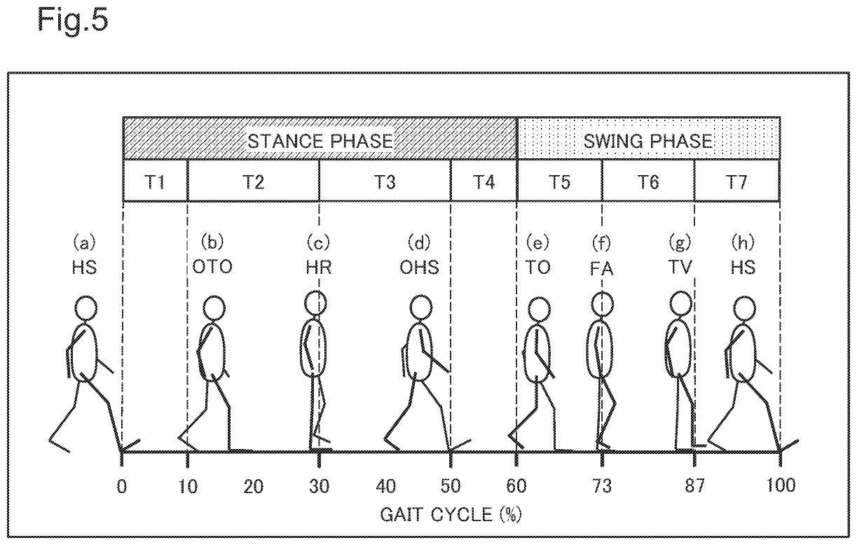

FIG. 5 is a conceptual diagram for explaining one gait cycle with the right foot as a reference. One gait cycle based on the left foot is also similar to that of the right foot. The horizontal axis of FIG. 5 is a normalized gait cycle with one gait cycle of the right foot as 100% with a time point at which the heel of the right foot lands on the ground as a starting point and a time point at which the heel of the right foot lands on the ground next as an ending point. The one gait cycle of one foot is roughly divided into a stance phase in which at least part of the back side of the foot is in contact with the ground and a swing phase in which the back side of the foot is away from the ground. The stance phase is further subdivided into an initial stance period T1, a mid-stance period T2 of standing, a terminal stance period T3 of standing, and a pre-swing period T4. The swing phase is further subdivided into an initial swing period T5, a mid-swing period T6, and a terminal swing period T7. FIG. 5 is an example, and does not limit the periods constituting one gait cycle, the names of these periods, and the like.

As illustrated in FIG. 5, in gait, a plurality of events (also referred to as gait events) occur. (a) of FIG. 5 represents an event (heel strike (HS)) in which the heel of the right foot is grounded. (b) of FIG. 5 illustrates an event (opposite toe off (OTO)) in which the toe of the right foot is away from the ground while the sole of the left foot is grounded. (c) of FIG. 5 illustrates an event (heel rise (HR)) in which the heel of the right foot is lifted while the sole of the right foot is grounded. (d) of FIG. 5 is an event (opposite heel strike (OHS)) in which the heel of the left foot is grounded. (e) of FIG. 5 illustrates an event (toe off (TO)) in which the toe of the left foot is away from the ground while the sole of the right foot is grounded. (f) of FIG. 5 illustrates an event (foot adjacent (FA)) in which the left foot and the right foot cross each other while the sole of the left foot is grounded. (g) of FIG. 5 illustrates an event (tibia vertical (TV)) in which the tibia of the left foot is substantially perpendicular to the ground while the sole of the right foot is grounded. (h) of FIG. 5 represents an event (heel strike (HS)) in which the heel of the right foot is grounded. (h) of FIG. 5 corresponds to the ending point of the gait cycle starting from (a) of FIG. 5 and corresponds to the starting point of the next gait cycle. FIG. 5 is an example, and does not limit events that occur during gait or names of these events.

[Data Acquisition Device]

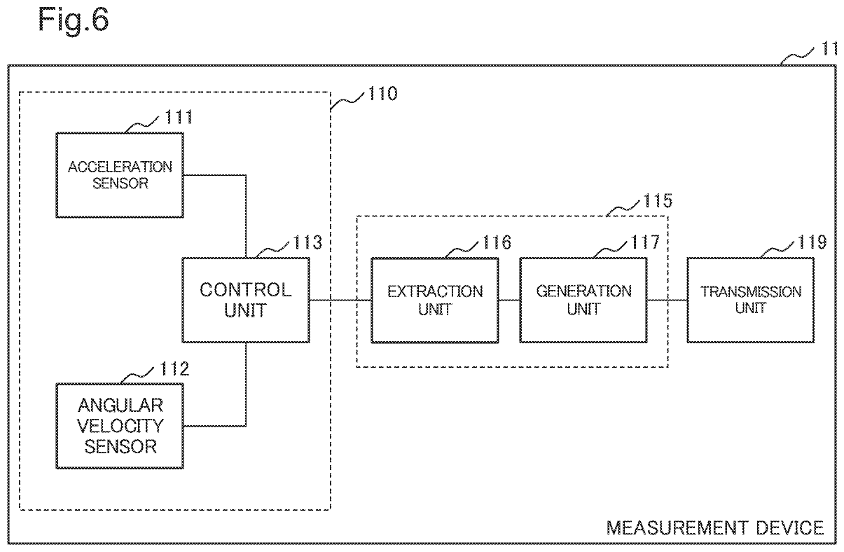

Next, the measurement device 11 will be described in detail with reference to the drawings. FIG. 6 is a block diagram illustrating an example of a detailed configuration of the measurement device 11. The measurement device 11 includes an acceleration sensor 111, an angular velocity sensor 112, a control unit 113, an extraction unit 116, a generation unit 117, and a transmission unit 119. The measurement device 11 includes a power supply not illustrated. The acceleration sensor 111, the angular velocity sensor 112, and the control unit 113 constitute a data acquisition unit 110 (also referred to as a data acquisition device). The extraction unit 116 and the generation unit 117 constitute a feature-amount generation unit 115 (also referred to as a feature-amount generation device).

The acceleration sensor 111 is a sensor that measures acceleration (also referred to as spatial acceleration) in the three axial directions. The acceleration sensor 111 outputs the measured acceleration to the control unit 113. For example, a sensor of a piezoelectric type, a piezoresistive type, a capacitance type, or the like can be used as the acceleration sensor 111. As long as the sensor used for the acceleration sensor 111 can measure an acceleration, the measurement method is not limited.

The angular velocity sensor 112 is a sensor that measures angular velocities around the three axes (also referred to as spatial angular velocities). Angular velocity sensor 112 outputs the measured angular velocity to control unit 113. For example, a sensor of a vibration type, a capacitance type, or the like can be used as the angular velocity sensor 112. As long as the sensor used for the angular velocity sensor 112 can measure an angular velocity, the measurement method is not limited.

The control unit 113 acquires an actual measurement value of acceleration in three axial directions from the acceleration sensor 111. The control unit 113 acquires an actual measurement value of the angular velocity around the axis from the angular velocity sensor 112. The control unit 113 converts the acquired actual measurement values of the acceleration and the angular velocity into digital data (also referred to as sensor data). The control unit 113 outputs the converted digital data to the extraction unit 116. The sensor data includes at least acceleration data (including acceleration vectors in three axial directions) and angular velocity data (including angular velocity vectors around the three axes) converted into digital data. The sensor data includes an acquisition time of an actual measurement value on which the acceleration data and the angular velocity data are based. The control unit 113 may be configured to output sensor data obtained by adding correction such as a mounting error, temperature correction, and linearity correction to the acquired acceleration data and angular velocity data. The control unit 113 may generate angle data (also referred to as a plantar angle) around three axes using the acquired acceleration data and angular velocity data.

For example, the control unit 113 is a microcomputer or a microcontroller that performs overall control and data processing of the measurement device 11. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantity (analog data) measured by each of the acceleration sensor 111 and the angular velocity sensor 112 may be converted into digital data in each of the acceleration sensor 111 and the angular velocity sensor 112. The digital data stored in the flash memory is output to the transmission unit 119 at a prescribed timing.

The extraction unit 116 acquires sensor data from the control unit 113. The extraction unit 116 generates a gait waveform for one gait cycle from the time-series data of the sensor data. For example, the extraction unit 116 generates a gait waveform related to acceleration in three axial directions, angular velocities around the three axes, and a plantar angle. In the present example embodiment, the extraction unit 116 generates a gait waveform for one gait cycle with the heel strike as a starting point. The extraction unit 116 extracts a feature amount (first feature amount) from a gait waveform for one gait cycle. When the feature amount is extracted from each of the temporally continuous gait phases, the extraction unit 116 integrates these gait phases to extract a gait phase cluster. For example, the extraction unit 116 may be configured to extract the feature amount from the gait phase cluster related to the physical feature of the measurement target based on a preset condition. For example, the extraction unit 116 extracts a feature amount related to a gait affected by a physical feature. The extraction unit 116 outputs the extracted feature amount to the generation unit 117.

The generation unit 117 acquires a feature amount (first feature amount) from the extraction unit 116. The generation unit 117 generates the feature amount (second feature amount) of the gait phase cluster using the feature-amount constitutive expression. The generation unit 117 generates feature-amount data in which gait phases (gait cycle) constituting a gait phase cluster and a feature amount of the gait phase cluster are associated with each other. In a case where the feature amount is extracted from a single gait phase, the generation unit 117 generates feature-amount data in which the extracted feature amount is associated with the gait phase. The generation unit 117 outputs the generated feature-amount data of the gait phase cluster to the transmission unit 119.

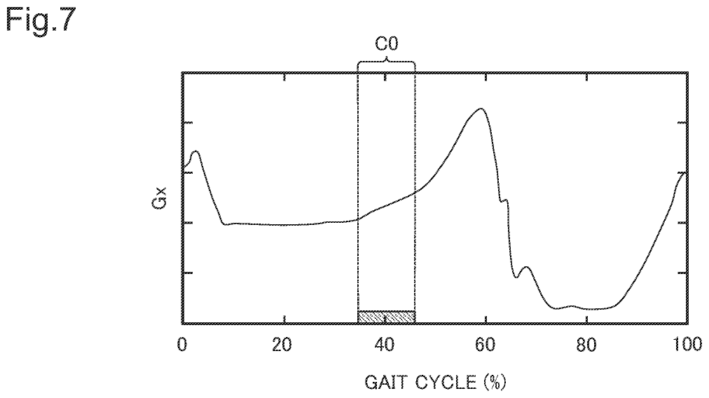

FIG. 7 is a graph for explaining a gait phase cluster. The graph of FIG. 7 is an example of a gait waveform Gx of the plantar angle around the x-axis. The gait waveform Gx is a waveform for one gait cycle (0 to 100%) with the heel strike as a starting point. In the example of FIG. 7, the feature amount (first feature amount) is extracted by the extraction unit 116 from each of the continuous gait phases in which the gait cycle is around 40%. The continuous gait phases having a gait cycle of around 40% are a gait phase cluster C0. The generation unit 117 applies the feature-amount constitutive expression to the feature amount extracted from each of the gait phases constituting the gait phase cluster to generate the feature amount (second feature amount) of the gait phase cluster C0.

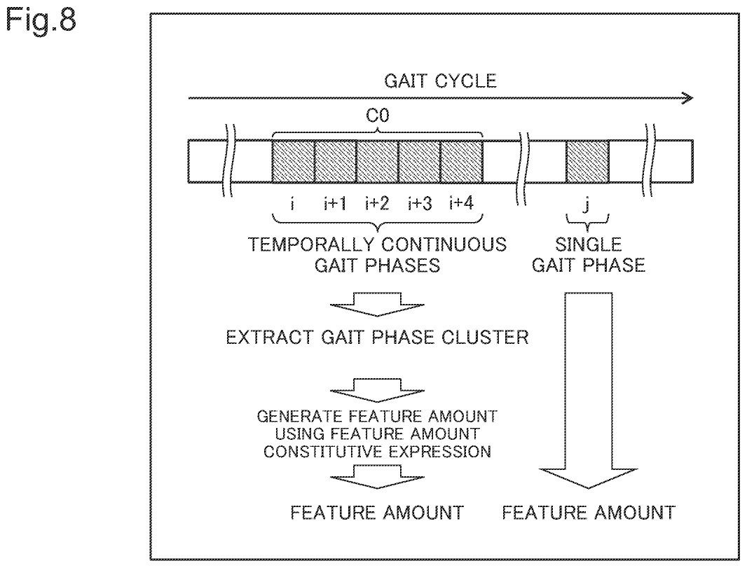

FIG. 8 is a conceptual diagram for describing extraction of a feature amount from a gait waveform for one gait cycle. The extraction unit 116 extracts temporally continuous gait phases i to i+4 as the gait phase cluster C0 (i is a natural number). The extraction unit 116 extracts a feature amount from each of the gait phases i to i+4. The generation unit 117 applies the feature-amount constitutive expression to the feature amount (first feature amount) extracted from each of the gait phases i to i+4 constituting the gait phase cluster C0 to generate the feature amount (second feature amount) of the gait phase cluster C0. The generation unit 117 generates feature-amount data in which the generated feature amount of the gait phase cluster C0 is associated with the gait cycle of the gait cycle phase cluster. The extraction unit 116 extracts a feature amount (first feature amount) from the single gait phase j. The generation unit 117 generates feature-amount data in which the feature amount extracted from the single gait phase j is associated with the gait cycle of the gait phase. The feature-amount constitutive expression may be applied without distinguishing the gait phases i to i+4 and the single gait phase j constituting the gait phase cluster. For example, in a case where the feature-amount constitutive expression is applied to the single gait phase j, the inclination and the variation cannot be calculated, and thus the feature-amount constitutive expression for calculating the integral average, the arithmetic average, or the like may be used.

The transmission unit 119 acquires the feature-amount data from the generation unit 117. The transmission unit 119 transmits the acquired feature-amount data to the data processing device 15. The transmission unit 119 may transmit the feature-amount data to the data processing device 15 via a wire such as a cable, or may transmit the feature-amount data to the data processing device 15 via wireless communication. For example, the transmission unit 119 is configured to transmit the feature-amount data to the data processing device 15 via a wireless communication function (not illustrated) conforming to a standard such as Bluetooth (registered trademark) or WiFi (registered trademark). The communication function of the transmission unit 119 may conform to a standard other than Bluetooth (registered trademark) or WiFi (registered trademark).

[Data Processing Device]

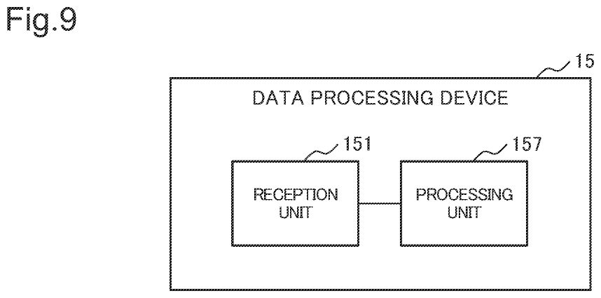

Next, details of the data processing device 15 will be described with reference to the drawings. FIG. 9 is a block diagram illustrating an example of a detailed configuration of the data processing device 15. The data processing device 15 includes a reception unit 151 and a processing unit 157.

The reception unit 151 receives the feature-amount data from the measurement device 11. The reception unit 151 outputs the received feature-amount data to the processing unit 157. The reception unit 151 may receive the feature-amount data from the measurement device 11 via a wire such as a cable, or may receive the feature-amount data from the measurement device 11 via wireless communication. For example, the reception unit 151 is configured to receive the feature-amount data from the measurement device 11 via a wireless communication function (not illustrated) conforming to a standard such as Bluetooth (registered trademark) or WiFi (registered trademark). The communication function of the reception unit 151 may conform to a standard other than Bluetooth (registered trademark) or WiFi (registered trademark).

The processing unit 157 acquires the feature-amount data from the reception unit 151. The processing unit 157 performs set data processing using the acquired feature-amount data. For example, the processing unit 157 estimates the physical feature of the user using the received feature-amount data. For example, the processing unit 157 inputs the feature-amount data to an estimation model prepared in advance, and estimates the physical feature of the user according to the output value. For example, the estimation model is a model for estimating physical feature such as the progress status of hallux valgus and the degree of pronation/supination. The processing unit 157 may perform any data processing as long as the feature-amount data acquired from the measurement device 11 is used. The processing unit 157 outputs a result of the data processing. For example, the processing unit 157 outputs a result of the data processing to a display device (not illustrated). The result of the data processing output to the display device is displayed on the screen of the display device. For example, the processing unit 157 outputs a result of the data processing to an external system. The result of the data processing output to the external system is used for any purpose.

(Operation)

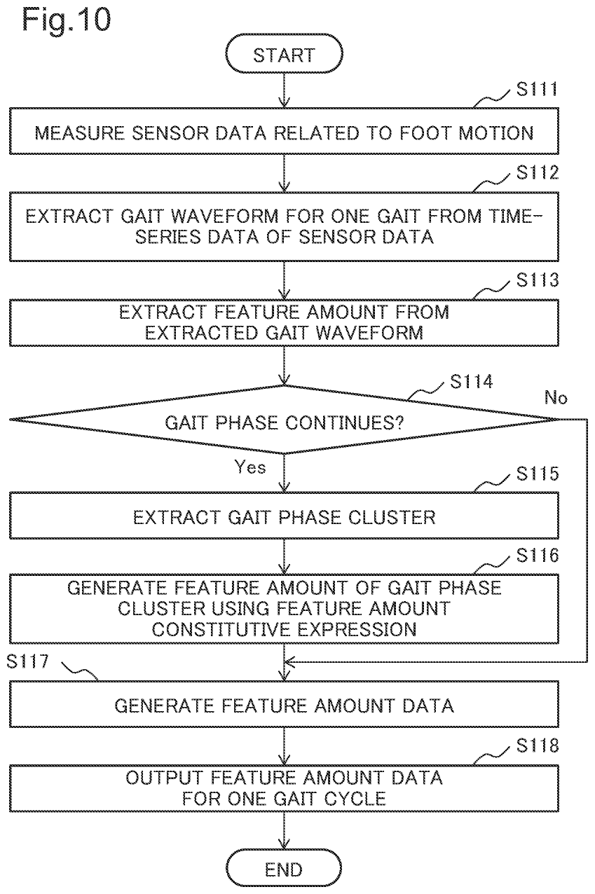

Next, an operation of the gait measurement system 10 will be described with reference to the drawings. An operation of the measurement device 11 included in the gait measurement system 10 will be described. FIG. 10 is a flowchart for explaining the operation of the measurement device 11. In the description along the flowchart of FIG. 10, the measurement device 11 will be described as an operation subject.

In FIG. 10, first, the measurement device 11 measures sensor data related to a motion of the foot (step S111).

Next, the measurement device 11 extracts a gait waveform for one gait cycle from the time-series data of the sensor data (step S112).

Next, the measurement device 11 extracts a feature amount (first feature amount) from the extracted gait waveform (step S113).

When the gait phases from each of which the feature amount is extracted are temporally continuous (Yes in step S114), the measurement device 11 extracts the continuous gait phases as a gait phase cluster (step S115). In a case where the gait phases from each of which the feature amount is extracted are not temporally continuous (No in step S114), the process proceeds to step S117. When the single gait phase is also processed as the gait phase cluster, step S114 may be omitted. In this case, the measurement device 11 regards the feature amount extracted from the single gait phase as the second feature amount and performs processing.

After step S115, the measurement device 11 generates the feature amount (second feature amount) of the gait phase cluster using the feature-amount constitutive expression (step S116).

Next, the measurement device 11 generates feature-amount data in which the generated feature amount of the gait phase cluster is associated with the gait cycle from which the gait phase cluster is extracted (step S117). In the case next to No in step S114, the measurement device 11 generates the feature-amount data associated with the gait cycle of the gait phase using the feature amount extracted from the single gait phase. In this case, the measurement device 11 regards the feature amount extracted from the single gait phase as the second feature amount and generates the feature-amount data.

Next, the measurement device 11 outputs the generated feature-amount data for one gait cycle to the data processing device 15 (step S118). For example, the feature amount output to the data processing device 15 is used for data processing such as physical feature estimation.

As described above, the gait measurement system of the present example embodiment includes the measurement device and the data processing device. The measurement device includes a data acquisition unit, a feature-amount generation unit, and a transmission unit. The data acquisition unit includes an acceleration sensor, an angular velocity sensor, and a control unit. The measurement device is disposed at footwear, of a user, to be measured. The data acquisition unit measures the spatial acceleration and the spatial angular velocity according to the gait of the user. The data acquisition unit generates sensor data based on the measured spatial acceleration and spatial angular velocity. The data acquisition unit outputs the generated sensor data to the feature-amount generation unit. The feature-amount generation unit includes an extraction unit and a generation unit. The extraction unit generates a gait waveform for one gait cycle from the time-series data of the sensor data related to a motion of the foot. The extraction unit extracts a feature amount (first feature amount) from the generated gait waveform. The extraction unit extracts a gait phase cluster by integrating a plurality of temporally continuous gait phases from each of which the feature amount (first feature amount) is extracted. The generation unit generates the feature amount (second feature amount) of the gait phase cluster using a preset feature-amount constitutive expression. The generation unit generates the feature-amount data in which the plurality of gait phases constituting the gait phase cluster is associated with the feature amount (second feature amount) of the gait phase cluster. The measurement device transmits the feature-amount data including the feature amount (second feature amount) of the gait phase cluster extracted by the feature-amount generation unit to the data processing device. The data processing device receives the feature amount (second feature amount) of the gait phase cluster transmitted by the feature-amount generation device. The data processing device performs data processing using the received feature amount (second feature amount) of the gait phase cluster.

According to the present example embodiment, it is possible to reduce the amount of data for verifying the feature appearing in the gait by generating the feature amount of the gait phase cluster obtained by integrating a plurality of temporally continuous gait phases. According to the present example embodiment, since the amount of data transmitted from the edge device (measurement device) can be reduced, the communication capacity of the edge device can be reduced.

Generally, due to the temporal continuity of the gait motion, the physical features appear not in a single gait phase (gait cycle), but in a series of continuous gait phases. In the present example embodiment, a series of gait phases are integrated as a gait phase cluster, and a single feature amount is generated from the gait phase cluster. Therefore, according to the present example embodiment, by extracting a single feature amount for each gait phase cluster affected by the physical feature, the physical feature can be accurately estimated even when the number of feature amounts to be verified is reduced.

In an aspect of the present example embodiment, in a case where the feature amount is extracted from the single gait phase that is not temporally continuous, the extraction unit outputs the feature amount extracted from the single gait phase to the generation unit. The generation unit generates feature-amount data in which a single gait phase and a feature amount of the single gait phase are associated with each other. In the present aspect, a single gait phase that is not temporally continuous is not extracted as a gait phase cluster. Therefore, according to the present aspect, for the single gait phase, the process of extracting the gait phase cluster can be reduced.

In an aspect of the present example embodiment, when a feature amount is extracted from a single gait phase that is not temporally continuous, the extraction unit extracts the single gait phase as a gait phase cluster. The generation unit generates the feature-amount data in which the single gait phase extracted as the gait phase cluster is associated with the feature amount of the single gait phase. In the present aspect, a single gait phase that is not temporally continuous is extracted as a gait phase cluster. Therefore, according to the present aspect, it is possible to perform processing without distinguishing between a plurality of temporally continuous gait phases and a single gait phase that is not temporally continuous.

In an aspect of the present example embodiment, the extraction unit extracts a feature amount of each of gait phases constituting a preset gait phase cluster to be extracted. According to the present example embodiment, since the feature amount to be extracted is limited, the processing of extracting the feature amount can be simplified.

In an aspect of the present example embodiment, the extraction unit extracts a feature amount related to a gait affected by a specific physical feature. According to the present example embodiment, since the feature amount related to the gait affected by the intended physical feature is extracted, the feature amount with high usability can be provided. According to the present example embodiment, since the feature amount to be extracted is limited, the processing of extracting the feature amount can be simplified.

### Second Example Embodiment

Next, a gait measurement system according to the second example embodiment will be described with reference to the drawings. The gait measurement system of the present example embodiment transmits sensor data measured by the sensor to the data processing unit, and extracts a feature amount from the sensor data by the data processing unit. Although the gait measurement system of the present example embodiment can be used in daily use, it has a configuration in which there is no restriction on the data communication amount of sensor data, and it is suitable for an environment in which sensor data is processed in a cloud.

(Configuration)

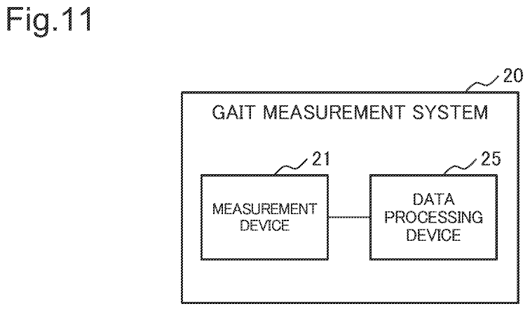

FIG. 11 is a block diagram illustrating an example of a configuration of a gait measurement system 20 of the present example embodiment. The gait measurement system 20 includes a measurement device 21 and a data processing device 25. The measurement device 21 and the data processing device 25 may be connected by wire or wirelessly. The measurement device 21 and the data processing device 25 may be configured by a single device. The measurement device 21 may be removed from the configuration of the gait measurement system 20, and the gait measurement system 20 may be configured only by the data processing device 25. Although only one measurement device 21 is illustrated in FIG. 11, one measurement device 21 (two in total) may be disposed at each of the left and right feet.

The measurement device 21 is installed at at least one of the left and right feet. The measurement device 21 is installed at footwear such as shoes, as in the measurement device 11 of the first example embodiment. The measurement device 21 includes an acceleration sensor and an angular velocity sensor. The measurement device 21 measures, as a physical quantity related to a motion of the foot of the user wearing the footwear, a physical quantity related to a motion of the foot such as acceleration in three axial directions (also referred to as spatial acceleration) and angular velocities around the three axes (also referred to as spatial angular velocities). The physical quantity related to a motion of the foot measured by the measurement device 21 includes not only the acceleration and the angular velocity but also the velocity and the angle calculated by integrating the acceleration and the angular velocity. The physical quantity related to a motion of the foot measured by the measurement device 21 also includes a position (trajectory) calculated by second-order integration of the acceleration.

The measurement device 21 converts the measured physical quantity into digital data (also referred to as sensor data). The measurement device 21 transmits the converted sensor data to the data processing device 25. For example, as in the measurement device 11 of the first example embodiment, the measurement device 21 is connected to the data processing device 25 via a mobile terminal (not illustrated) carried by the user.

The data processing device 25 receives sensor data from the measurement device 21. The data processing device 25 generates a waveform (also referred to as a gait waveform) for one gait cycle from the time-series data of the sensor data. For example, the data processing device 25 generates a gait waveform related to acceleration in three axial directions and angular velocities around the three axes. For example, the data processing device 25 generates a gait waveform related to the plantar angle using the acceleration in three axial directions and the angular velocities around the three axes. The data processing device 25 extracts a feature amount related to the physical feature of the user from the gait waveform for one gait cycle. The data processing device 25 generates a feature amount of temporally continuous gait phases (also referred to as a gait phase cluster) using the feature-amount constitutive expression. The data processing device 25 generates feature-amount data in which gait phases (gait cycle) constituting a gait phase cluster and a feature amount of the gait phase cluster are associated with each other. The data processing device 25 performs set data processing using the generated feature-amount data. The data processing device 25 may perform any data processing using the generated feature-amount data. The data processing device 25 outputs a result of the data processing. For example, the data processing device 25 outputs a result of the data processing to a display device (not illustrated) or an external system.

[Data Acquisition Device]

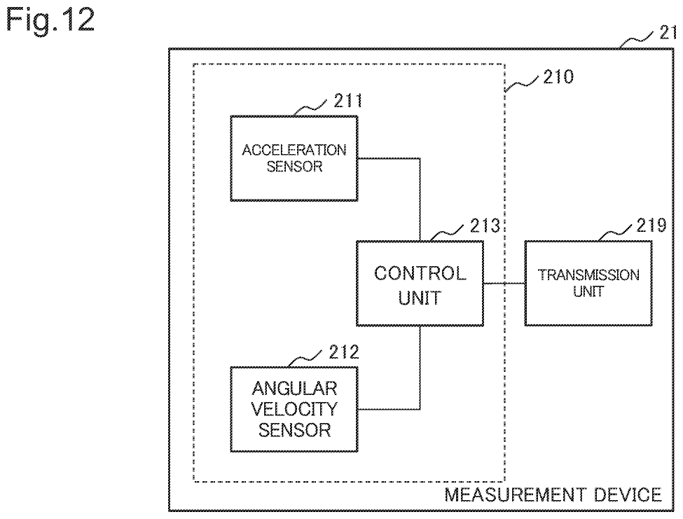

Next, the measurement device 21 will be described in detail with reference to the drawings. FIG. 12 is a block diagram illustrating an example of a detailed configuration of the measurement device 21. The measurement device 21 includes an acceleration sensor 211, an angular velocity sensor 212, a control unit 213, and a transmission unit 219. The measurement device 21 includes a power supply not illustrated. The acceleration sensor 211, the angular velocity sensor 212, and the control unit 213 constitute a data acquisition unit 210 (data acquisition device).

The acceleration sensor 211 is a sensor that measures acceleration (also referred to as spatial accelerations) in the three axial directions. The acceleration sensor 211 outputs the measured acceleration to the control unit 213. The acceleration sensor 211 has a configuration similar to that of the acceleration sensor 111 of the first example embodiment.

The angular velocity sensor 212 is a sensor that measures angular velocities (also referred to as spatial angular velocities) around three axes. Angular velocity sensor 212 outputs the measured angular velocity to control unit 213. The angular velocity sensor 212 has a configuration similar to that of the angular velocity sensor 112 of the first example embodiment.

The control unit 213 acquires actual measurement values of the acceleration in three axial directions and the angular velocities around the three axes from the acceleration sensor 211 and the angular velocity sensor 212. The control unit 213 has a configuration similar to that of the control unit 113 of the first example embodiment. The control unit 213 converts the acquired actual measurement values of the acceleration and the angular velocity into digital data (also referred to as sensor data). The control unit 213 outputs the converted digital data to a transmission unit 219.

The transmission unit 219 acquires data of the control unit 213. The transmission unit 219 has a configuration similar to that of the transmission unit 119 of the first example embodiment. The transmission unit 219 transmits the acquired sensor data to the data processing device 15.

[Data Processing Device]

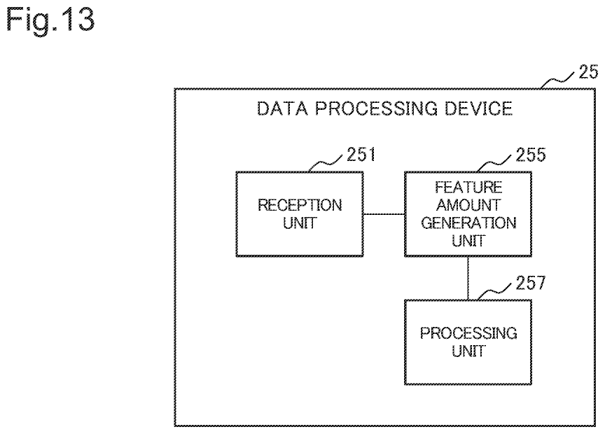

Next, details of the data processing device 25 will be described with reference to the drawings. FIG. 13 is a block diagram illustrating an example of a detailed configuration of the data processing device 25. The data processing device 25 includes a reception unit 251, a feature-amount generation unit 255, and a processing unit 257.

The reception unit 251 receives sensor data from the measurement device 21. The reception unit 251 has a configuration similar to that of the reception unit 151 of the first example embodiment. The reception unit 251 outputs the received feature-amount data to the feature-amount generation unit 255.

The feature-amount generation unit 255 (also referred to as a feature-amount generation device) acquires sensor data from the reception unit 251. The feature-amount generation unit 255 has a configuration similar to that of the feature-amount generation unit 115 including the extraction unit 116 and the generation unit 117 of the first example embodiment. The feature-amount generation unit 255 generates a gait waveform for one gait cycle from the time-series data of the sensor data. The feature-amount generation unit 255 extracts a feature amount (first feature amount) from a gait waveform for one gait cycle. When the feature amount is extracted from each of the temporally continuous gait phases, the feature-amount generation unit 255 extracts the gait phases as a gait phase cluster. The feature-amount generation unit 255 generates the feature amount (second feature amount) of the gait phase cluster using the feature-amount constitutive expression. The feature-amount generation unit 255 generates feature-amount data in which gait phases (gait cycle) constituting a gait phase cluster and a feature amount of the gait phase cluster are associated with each other. In a case where the feature amount is extracted from a single gait phase, the feature-amount generation unit 255 generates feature-amount data in which the extracted feature amount is associated with the gait phase. The feature-amount generation unit 255 outputs the generated feature-amount data of the gait phase cluster to the processing unit 257.

The processing unit 257 acquires the feature-amount data from the feature-amount generation unit 255. The processing unit 257 has a configuration similar to that of the processing unit 157 of the first example embodiment. The processing unit 257 performs set data processing using the acquired feature-amount data. The processing unit 257 may perform any data processing as long as the feature-amount data acquired from the feature-amount generation unit 255 is used. The processing unit 257 outputs a result of the data processing. For example, the processing unit 257 outputs a result of the data processing to a display device (not illustrated) or an external system.

As described above, the gait measurement system of the present example embodiment includes the measurement device and the data processing device. The measurement device includes a data acquisition unit and a transmission unit. The data acquisition unit includes an acceleration sensor, an angular velocity sensor, and a control unit. The measurement device is disposed at footwear, of a user, to be measured. The data acquisition unit measures the spatial acceleration and the spatial angular velocity according to the gait of the user. The data acquisition unit generates sensor data based on the measured spatial acceleration and spatial angular velocity. The data acquisition unit transmits the generated sensor data to the data processing device. The data processing device includes a reception unit, a feature-amount generation unit, and a processing unit. The reception unit receives sensor data from the measurement device. The feature-amount generation unit generates a gait waveform for one gait cycle from the received time-series data of the sensor data. The feature-amount generation unit extracts a feature amount (first feature amount) from the generated gait waveform. The feature-amount generation unit extracts a gait phase cluster by integrating a plurality of temporally continuous gait phases from each of which the feature amount (first feature amount) is extracted. The feature-amount generation unit generates a feature amount (second feature amount) of the gait phase cluster using a preset feature-amount constitutive expression. The feature-amount generation unit generates feature-amount data in which a plurality of gait phases constituting a gait phase cluster is associated with a feature amount (second feature amount) of the gait phase cluster. The feature-amount generation unit performs data processing using the generated feature amount (second feature amount) of the gait phase cluster.

According to the present example embodiment, it is possible to reduce the amount of data for verifying the feature appearing in the gait by generating the feature amount of the gait phase cluster obtained by integrating a plurality of temporally continuous gait phases. Therefore, according to the present example embodiment, the load of data processing using the feature amount is reduced.

### Third Example Embodiment

Next, a training system according to the third example embodiment will be described with reference to the drawings. The training system of the present example embodiment generates an estimation model for estimating a physical feature according to an input of a feature amount by training using the feature amount extracted from sensor data measured by a measurement device.

(Configuration)

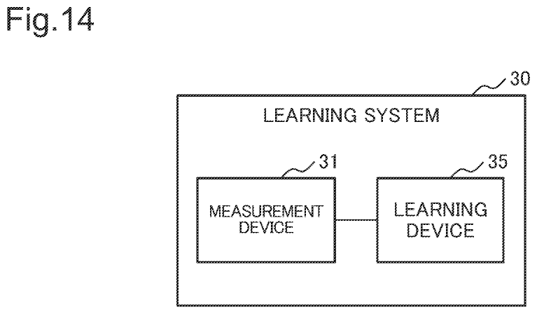

FIG. 14 is a block diagram illustrating an example of a configuration of a training system 30 of the present example embodiment. The training system 30 includes a measurement device 31 and a training device 35. The measurement device 31 and the training device 35 may be connected by wire or wirelessly. The measurement device 31 and the training device 35 may be configured by a single device. The training system 30 may be configured only by the training device 35 by excluding the measurement device 31 from the configuration of the training system 30. Although only one measurement device 31 is illustrated in FIG. 14, one measurement device 31 (two in total) may be disposed at each of the left and right feet. The training device 35 may be configured not to be connected to the measurement device 31 but to perform training using the feature-amount data generated in advance by the measurement device 31 and stored in the database.

The measurement device 31 is installed at at least one of the left and right feet. The measurement device 31 has a configuration similar to that of the measurement device 11 of the first example embodiment. The measurement device 31 includes an acceleration sensor and an angular velocity sensor. The measurement device 31 converts the measured physical quantity into digital data (also referred to as sensor data). The measurement device 31 generates a waveform (also referred to as a gait waveform) for one gait cycle from the time-series data of the sensor data. The measurement device 31 extracts a feature amount (first feature amount) from a gait waveform for one gait cycle. When the feature amount is extracted from each of the temporally continuous gait phases, the measurement device 31 extracts a cluster (also referred to as a gait phase cluster) by integrating the gait phases. The measurement device 31 generates the feature amount (second feature amount) of the gait phase cluster using the feature-amount constitutive expression. The measurement device 31 generates feature-amount data in which gait phases (gait cycle) constituting a gait phase cluster and a feature amount of the gait phase cluster are associated with each other. The measurement device 31 transmits the generated feature-amount data of the gait phase cluster to the training device 35. The measurement device 31 may be configured to transmit the feature-amount data to a database (not illustrated) accessed by the training device 35. The feature-amount data accumulated in the database is used for training by the training device 35.

The training device 35 receives the feature-amount data from the measurement device 31. When using the feature-amount data accumulated in the database (not illustrated), the training device 35 receives the feature-amount data from the database. The training device 35 performs training using the received feature-amount data. For example, the training device 35 performs training with the feature-amount data extracted from the gait waveforms of the plurality of users as training data. The training algorithm performed by the training device 35 is not particularly limited. The training device 35 generates an estimation model trained for a plurality of users. The training device 35 stores the generated estimation model. The estimation model trained by the training device 35 may be stored in a storage device outside the training device 35.

[Training Device]

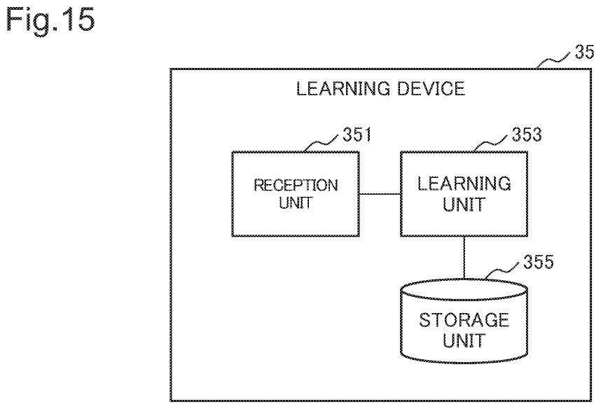

Next, details of the training device 35 will be described with reference to the drawings. FIG. 15 is a block diagram illustrating an example of a detailed configuration of the training device 35. The training device 35 includes a reception unit 351, a training unit 353, and a storage unit 355.

The reception unit 351 receives the feature-amount data from the measurement device 31. The reception unit 351 has a configuration similar to that of the measurement device 11 of the first example embodiment. The reception unit 351 outputs the received feature-amount data to the training unit 353.

The training unit 353 acquires the feature-amount data from the reception unit 351. The training unit 353 performs training unit using the acquired feature-amount data. For example, the training unit 353 perform training unit with a data set in which a feature amount extracted from a user having a certain physical feature is used as an explanatory variable and a physical feature of the user is used as an objective variable as training data. For example, the training unit 353 generates an estimation model that estimates a physical feature based on a feature amount extracted from sensor data detected with gait where the estimation model is trained for a plurality of users. For example, the training unit 353 stores the estimation model trained for a plurality of users in the storage unit 355.

For example, the training unit 353 performs training unit using a linear regression algorithm. It performs training unit using an algorithm of a support vector machine (SVM). For example, the training unit 353 performs training unit using a Gaussian process regression (GPR) algorithm. For example, the training unit 353 performs training unit using an algorithm such as random forest (RF). For example, the training unit 353 may perform unsupervised training unit that classifies a user who is a generation source of the feature-amount data according to the feature-amount data. The training unit algorithm performed by the training unit 353 is not particularly limited.

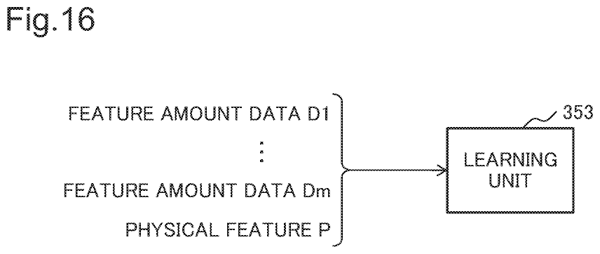

FIG. 16 is a conceptual diagram illustrating an example of causing the training unit 353 to perform training unit with a data set of feature-amount data D1 to Dm (m is a natural number) which are explanatory variables and a physical feature P which is an objective variable as training data. For example, the training unit 353 generates an estimation model that is trained with data related to a plurality of subjects, and that outputs a certain physical feature according to an input of a feature amount extracted from sensor data.

The storage unit 355 stores the estimation model trained for a plurality of subjects. For example, the storage unit 355 stores an estimation model that is trained for a plurality of subjects and that estimates a specific physical feature.

As described above, the training system of the present example embodiment includes the measurement device and the training device. The measurement device is disposed at footwear, of a user, to be measured. The measurement device measures the spatial acceleration and the spatial angular velocity according to the gait of the user. The measurement device generates sensor data based on the measured spatial acceleration and spatial angular velocity. The measurement device generates a gait waveform for one gait cycle from time-series data of sensor data related to a motion of a foot. The measurement device extracts a feature amount (first feature amount) from the generated gait waveform. The measurement device extracts a gait phase cluster by integrating a plurality of temporally continuous gait phases from each of which the feature amount is extracted. The measurement device generates a feature amount (second feature amount) of the gait phase cluster using a preset feature-amount constitutive expression. The measurement device generates the feature-amount data in which the plurality of gait phases constituting the gait phase cluster is associated with the feature amount (second feature amount) of the gait phase cluster.

The training device includes a reception unit, a training unit, and a storage unit. The reception unit acquires the feature-amount data generated by the measurement device. The training unit performs training unit using the feature-amount data. The training unit generates the estimation model that outputs the physical feature according to the input of the feature amount (second feature amount) of the gait phase cluster extracted from the time-series data of the sensor data measured along with the gait of the user. For example, the training unit generates the estimation model that outputs the degree of hallux valgus according to the input of the feature amount (second feature amount) of the gait phase cluster extracted from the time-series data of the sensor data measured along with the gait of the user. The estimation model generated by the training unit is stored in the storage unit.

According to the present example embodiment, the estimation model for estimating the physical feature can be generated using the feature amount of the gait phase cluster extracted according to the gait of the user. For example, according to the present example embodiment, the estimation model for estimating the degree of hallux valgus can be generated using the feature amount of the gait phase cluster extracted according to the gait of the user.

### Fourth Example Embodiment

Next, a gait measurement system according to the fourth example embodiment will be described with reference to the drawings. The gait measurement system of the present example embodiment estimates the physical feature of the user using the estimation model trained by the training system of the third example embodiment.

(Configuration)

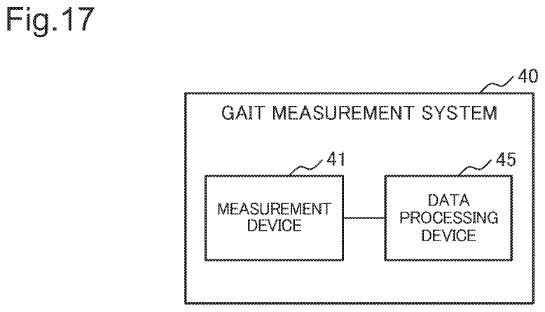

FIG. 17 is a block diagram illustrating an example of a configuration of a gait measurement system 40 of the present example embodiment. The gait measurement system 40 includes a measurement device 41 and a data processing device 45. The measurement device 41 and the data processing device 45 may be connected by wire or wirelessly. The measurement device 41 and the data processing device 45 may be configured by a single device. The measurement device 41 may be removed from the configuration of the gait measurement system 40, and the gait measurement system 40 may be configured only by the data processing device 45. Although only one measurement device 41 is illustrated in FIG. 17, one measurement device 41 (two in total) may be disposed at each of the left and right feet.

The measurement device 41 is installed at at least one of the left and right feet. The measurement device 41 has a configuration similar to that of the measurement device 11 of the first example embodiment. The measurement device 41 includes an acceleration sensor and an angular velocity sensor. The measurement device 41 converts the measured physical quantity into digital data (also referred to as sensor data). The measurement device 41 generates a waveform (also referred to as a gait waveform) for one gait cycle from the time-series data of the sensor data. The measurement device 41 extracts a feature amount (first feature amount) from a gait waveform for one gait cycle. When the feature amount is extracted from each of the temporally continuous gait phases, the measurement device 41 extracts a cluster (also referred to as a gait phase cluster) by integrating the gait phases. The measurement device 41 generates the feature amount (second feature amount) of the gait phase cluster using the feature-amount constitutive expression. The measurement device 41 generates feature-amount data in which gait phases (gait cycle) constituting a gait phase cluster and a feature amount (second feature amount) of the gait phase cluster are associated with each other. The measurement device 41 transmits the generated feature-amount data of the gait phase cluster to the data processing device 45.

The data processing device 45 receives the feature-amount data from the measurement device 41. The data processing device 45 inputs the received feature-amount data to an estimation model prepared in advance and performs estimation. For example, the data processing device 45 inputs feature-amount data to an estimation model that estimates a physical feature to estimate the physical feature of the user. The data processing device 45 outputs the estimation result. For example, the data processing device 45 outputs the estimation result to a display device (not illustrated) or an external system.

[Data Processing Device]

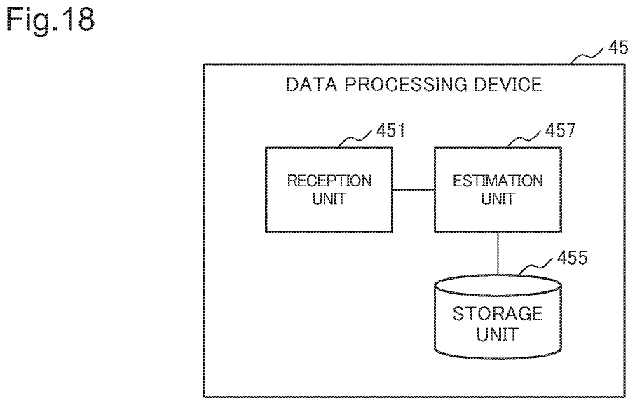

Next, details of the data processing device 45 will be described with reference to the drawings. FIG. 18 is a block diagram illustrating an example of a detailed configuration of the data processing device 45. The data processing device 45 includes a reception unit 451, a storage unit 455, and an estimation unit 457.

The reception unit 451 receives the feature-amount data from the measurement device 41. The reception unit 451 has a configuration similar to that of the measurement device 11 of the first example embodiment. The reception unit 451 outputs the received feature-amount data to the estimation unit 457.

The storage unit 455 stores the estimation model trained for a plurality of subjects. For example, the storage unit 455 stores an estimation model that is trained for a plurality of subjects and that estimates a certain physical feature. The estimation model may be stored in the storage unit 455 at the time of factory shipment of a product, calibration before the user uses the data processing device 45, or the like. In a case where an estimation model stored in a storage device such as an external server is used, the estimation model may be used via an interface (not illustrated) connected to the storage device. In this case, it is not necessary to store the estimation model in the storage unit 455.

The estimation unit 457 acquires the feature-amount data from the reception unit 451. The estimation unit 457 performs estimation using the acquired feature-amount data. The estimation unit 457 inputs the feature-amount data to the estimation model stored in the storage unit 455. The estimation unit 457 outputs the output from the estimation model as an estimation result. In a case where an estimation model stored in a storage device such as an external server is used, the estimation model may be used via an interface (not illustrated) connected to the storage device.

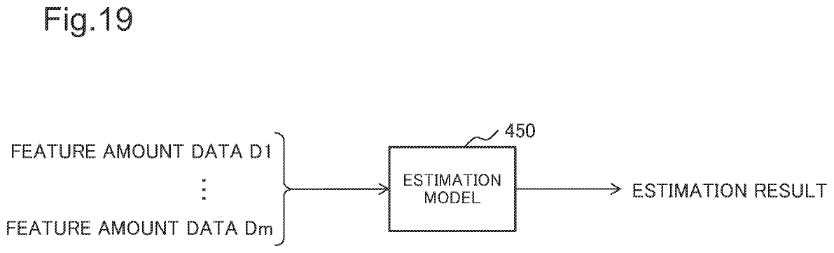

FIG. 19 is a conceptual diagram illustrating an example in which estimation results are output by inputting feature-amount data D1 to Dm (m is a natural number) including the feature amounts extracted from sensor data measured with the gait of a user to an estimation model 450 constructed in advance. For example, in the case of the estimation model 450 for estimating the degree of progress of hallux valgus, the degree of progress according to the input feature-amount data D1 to Dm is output from the estimation model 450. For example, in the case of the estimation model 450 for estimating the degree of pronation/supination of the foot, the degree related to the input feature-amount data D1 to Dm is output from the estimation model 450. The estimation result estimated by the estimation model 450 is not limited as long as the estimation result related to the physical feature can be output according to the input of the feature-amount data of the gait phase cluster.

[Application Example]

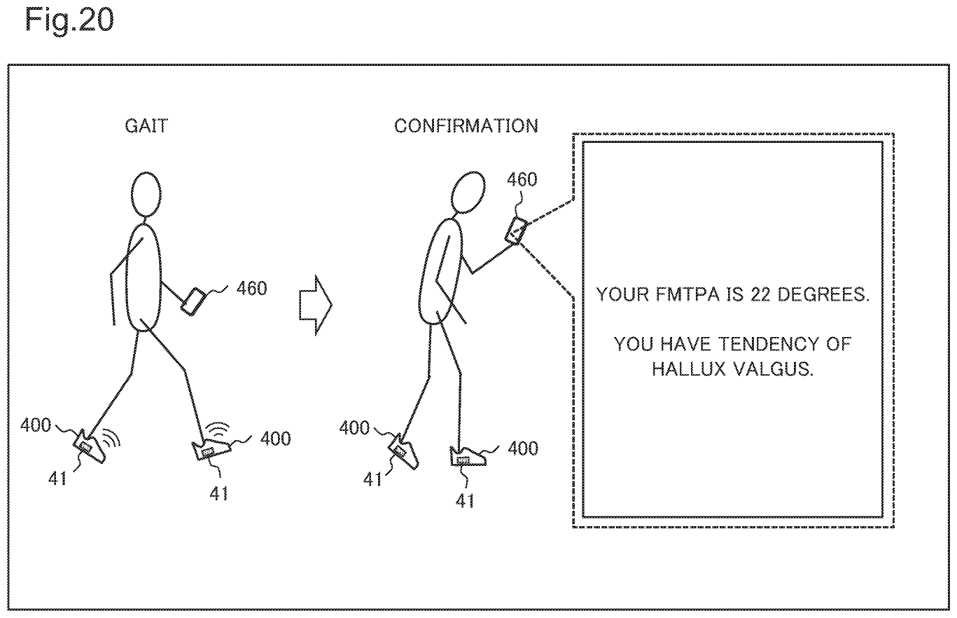

FIG. 20 is a conceptual diagram illustrating an example in which the estimation result by the estimation unit 457 of the data processing device 45 is displayed on the display unit of a mobile terminal 460 carried by the user walking while wearing footwear 400 at which the measurement device 41 is installed. FIG. 20 is an example in which information according to the estimation result using the feature-amount data accumulated while the user is walking is displayed on the display unit of the mobile terminal 460.

FIG. 20 illustrates an example in which the degree of progress of hallux valgus is displayed on the screen of the mobile terminal 460 according to the size of the first metatarsophalangeal angle (FMTPA). The FMTPA is an angle of the metatarsophalangeal joint of the first finger (thumb) of the foot. In the example of FIG. 20, based on the feature-amount data including the feature amount extracted from the sensor data measured while the user is walking, information that “Your FMTPA is 22 degrees. You have a tendency of hallux valgus” is displayed on the display unit of the mobile terminal 460. The user who has confirmed the information displayed on the display unit of the mobile terminal 460 can recognize the progress of his/her hallux valgus. For example, in a case where the degree of progression of hallux valgus is high, a message recommending an examination in a hospital or a contact address of an appropriate hospital may be displayed on the display unit of the mobile terminal 460.

FIG. 20 is an example, and does not limit a method of using the estimation result by the data processing device of the present example embodiment. For example, information about the gait such as the degree of pronation/supination of the left and right feet, the step length of the left and right feet, the trajectory of compasses, symmetry, and foot angle may be displayed on the display unit of the mobile terminal 460.

As described above, the gait measurement system of the present example embodiment includes the measurement device and the data processing device. The measurement device is disposed at footwear, of a user, to be measured. The measurement device measures the spatial acceleration and the spatial angular velocity according to the gait of the user. The measurement device generates sensor data based on the measured spatial acceleration and spatial angular velocity. The measurement device generates a gait waveform for one gait cycle from time-series data of sensor data related to a motion of a foot. The measurement device extracts a feature amount (first feature amount) from the generated gait waveform. The measurement device extracts a gait phase cluster by integrating a plurality of temporally continuous gait phases from each of which the feature amount is extracted. The measurement device generates a feature amount (second feature amount) of the gait phase cluster using a preset feature-amount constitutive expression. The measurement device generates the feature-amount data in which the plurality of gait phases constituting the gait phase cluster is associated with the feature amount (second feature amount) of the gait phase cluster. The measurement device transmits the feature-amount data including the feature amount (second feature amount) of the gait phase cluster to the data processing device.

The data processing device includes a reception unit, an estimation unit, and a storage unit. The reception unit acquires the feature-amount data generated by the measurement device. The storage unit stores an estimation model that outputs a physical feature according to the input feature amount. The estimation unit inputs the feature amount of the gait phase cluster extracted from the time-series data of the sensor data measured along with the gait of the user to the estimation model, and estimates the physical feature of the user based on the estimation value output from the estimation model. For example, the storage unit stores an estimation model that outputs the degree of hallux valgus according to the input feature amount. The estimation unit inputs the feature amount of the gait phase cluster extracted from the time-series data of the sensor data measured along with the gait of the user to the estimation model, and estimates the degree of hallux valgus of the user based on the estimation value output from the estimation model.

According to the present example embodiment, the physical feature of the user can be estimated using the feature amount of the gait phase cluster extracted according to the gait of the user. For example, according to the present example embodiment, the degree of hallux valgus can be estimated using the feature amount of the gait phase cluster extracted according to the gait of the user.

### Fifth Example Embodiment

Next, a feature-amount generation device according to the fifth example embodiment will be described with reference to the drawings. The feature-amount generation device of the present example embodiment has a configuration in which the feature-amount generation units of the first to fourth example embodiments are simplified.

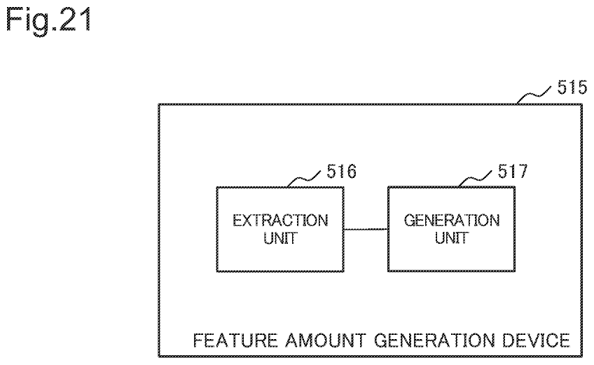

FIG. 21 is a block diagram illustrating an example of a configuration of a feature-amount generation device 515 according to the present example embodiment. The feature-amount generation device 515 includes an extraction unit 516 and a generation unit 517. The extraction unit 516 generates a gait waveform for one gait cycle from the time-series data of the sensor data related to a motion of the foot. The extraction unit 516 extracts a feature amount (first feature amount) from the generated gait waveform. The extraction unit 516 extracts a gait phase cluster by integrating a plurality of temporally continuous gait phases from each of which the feature amount is extracted. The generation unit 517 generates the feature amount (second feature amount) of the gait phase cluster using a preset feature-amount constitutive expression. The generation unit 517 generates the feature-amount data in which the plurality of gait phases constituting the gait phase cluster is associated with the feature amount (second feature amount) of the gait phase cluster.

According to the feature-amount generation device of the present example embodiment, it is possible to reduce the data amount for verifying the feature appearing in the gait by generating the feature amount of the gait phase cluster obtained by integrating a plurality of temporally continuous gait phases.

(Verification Example)

Next, a verification example in which the correlation of the gait waveform for one gait cycle normalized on the time axis with the degree of progress of hallux valgus is analyzed for each 1% gait phase (gait cycle) will be described with reference to the drawings. This verification example is an example of verifying the degree of progress of hallux valgus based on sensor data related to a motion of the foot. In this verification, nine types of gait waveforms (triaxial acceleration, triaxial angular velocity, triaxial plantar angle) were verified. In the following, a gait waveform from which a feature amount related to the degree of progression of hallux valgus is extracted is illustrated.

The degree of progression of hallux valgus is verified by a first metatarsophalangeal angle (FMTPA). The FMTPA is an angle of the metatarsophalangeal joint of the first finger (thumb) of the foot. In this verification example, in a case where the FMTPA exceeds 25 degrees, it is classified into hallux valgus. In a case where the FMTPA is equal to or greater than 15 degrees and equal to or less than 25 degrees, it is classified as a tendency of hallux valgus. In a case where the FMTPA less than 15 degrees, it is classified as normal. In this verification example, the correlation between the feature amount extracted from the gait waveform and the hallux valgus was analyzed using Pearson's correlation analysis. In this verification example, a gait phase (gait cycle) having significance (p<0.05) was recognized as a gait phase related to hallux valgus.

This validation was performed on 50 subjects. This verification was performed under the condition that the subject wearing the shoe at which the measurement device was installed walks at a comfortable speed without specifying the gait speed or the like. The measurement was performed in a sequence in which 50 subjects making a round trip 4 times over a distance of 8 meters under the same conditions. The 50 subjects were classified into Group A with the FMTPA greater than 25 degrees, Group B with the FMTPA equal to or greater than 15 degrees and equal to or less than 25 degrees, and Group C with the FMTPA less than 15 degrees. In the sequence of making round trip 4 times over a distance of 8 meters, sensor data for about 50 steps was acquired. Sensor data acquired from each subject was averaged according to the number of steps.

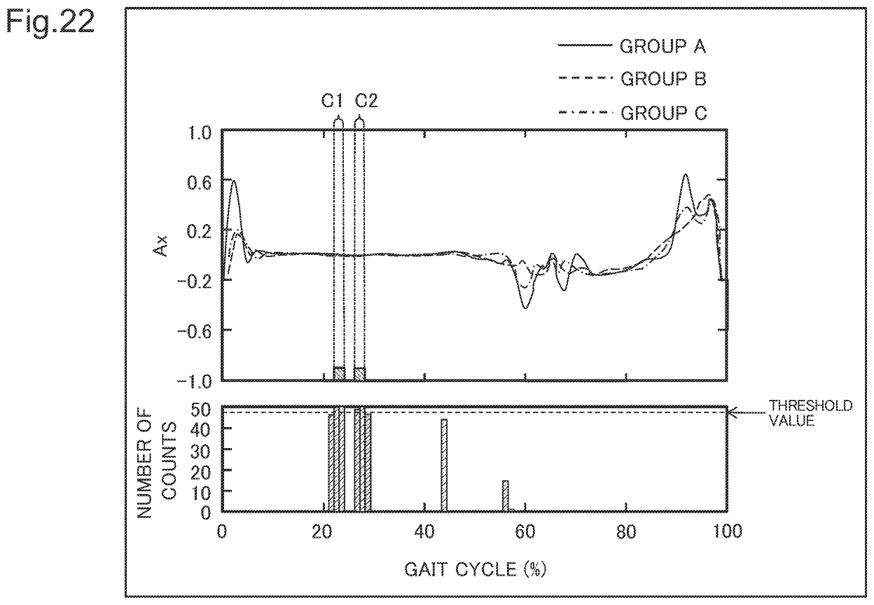

FIGS. 22 to 27 are diagrams in which a plurality of gait waveforms for one gait cycles from which feature amounts related to hallux valgus are extracted are associated with analysis results by correlation analysis of feature amount extracted from each of the gait waveforms. The gait waveform on the upper side in FIGS. 22 to 27 is a waveform for one gait cycle (0 to 100%) with the heel strike as a starting point. In the gait waveforms on the upper side in FIGS. 22 to 27, the waveform of the group A is indicated by a solid line, the waveform of the group B is indicated by a broken line, and the waveform of the group C is indicated by a one-dot chain line. The lower graphs in FIGS. 22 to 27 indicate the number (also referred to as a count number) recognized as having significance in correlation between hallux valgus and the feature amount by the Leave-one-subject-out correlation analysis. In the Leave-one-subject-out correlation analysis, individual difference factors are removed, and in order to verify whether the output value by the estimation model follows the essential distribution of data, correlation analysis is performed by sequentially excluding one subject. In this verification example, the correlation analysis using the feature-amount data for 49 subjects in which the feature-amount data of one subject is excluded from the feature-amount data of 50 subjects was repeated 50 times. In this verification, the threshold value of the count number is set to 47, and the feature amount of the count number of 47 or more is extracted. A feature amount with a count number of less than 47 was considered to have essentially no influence of hallux valgus. A feature amount having a count number of less than 47 causes a low correlation, and thus was excluded from the feature amount of the gait phase cluster and was not extracted. The threshold value of the count number may be set according to the purpose.

FIG. 22 illustrates an example in which the feature amount is extracted from the gait waveform of the x-direction acceleration. The upper graph of FIG. 22 is a gait waveform Ax of the x-direction acceleration. The gait waveform Ax is normalized so that 50 subjects can be compared under common conditions. In the example of FIG. 22, two feature amounts exceeding the threshold value of the count number are extracted among the plurality of extracted feature amounts. The first feature amount is a feature amount of the gait phase cluster C1 having a gait cycle of 22 to 24%. The gait phase cluster C1 corresponds to the early stage of the mid-stance period. In the early stage of the mid-stance period, the sole is grounded. The second feature amount is a feature amount of the gait phase cluster C2 having a gait cycle of 27 to 29%. The gait phase cluster C2 corresponds to the final stage of the mid-stance period. In the final stage of the mid-stance period leg, the sole is in contact with the ground.

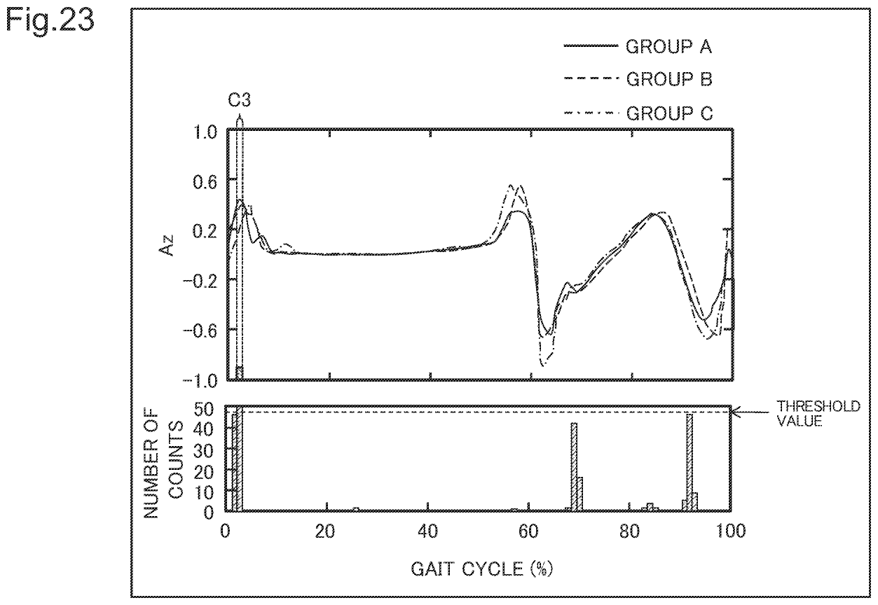

FIG. 23 illustrates an example in which the feature amount is extracted from the gait waveform of the z-direction acceleration. The upper graph of FIG. 23 is a gait waveform Az of the z-direction acceleration. The gait waveform Az is normalized so that 50 subjects can be compared under common conditions. In the example of FIG. 23, one feature amount exceeding the threshold value of the count number is extracted among the plurality of extracted feature amounts. The extracted feature amount is a feature amount of the gait phase cluster C3 having a gait cycle of 3 to 4%. The gait phase cluster C3 is a feature amount extracted from a single gait phase, but is regarded as a cluster. The gait phase cluster C3 corresponds to a period immediately after the heel strike.

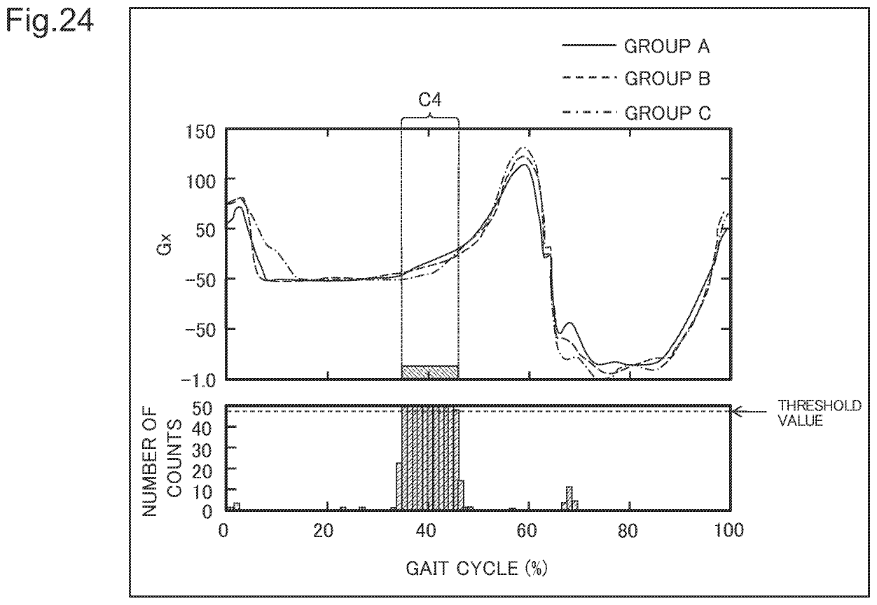

FIG. 24 illustrates an example in which the feature amount is extracted from the gait waveform of the angular velocity around the x-axis. The upper graph of FIG. 24 is a gait waveform Gx of the angular velocity around the x-axis. The gait waveform Gx is normalized so that 50 subjects can be compared under common conditions. In the example of FIG. 24, one feature amount exceeding the threshold value of the count number is extracted among the plurality of extracted feature amounts. The extracted feature amount is a feature amount of the gait phase cluster C4 having a gait cycle of 35 to 46%. The gait phase cluster C4 corresponds to the terminal stance period. The terminal stance period includes the timing of heel rise.

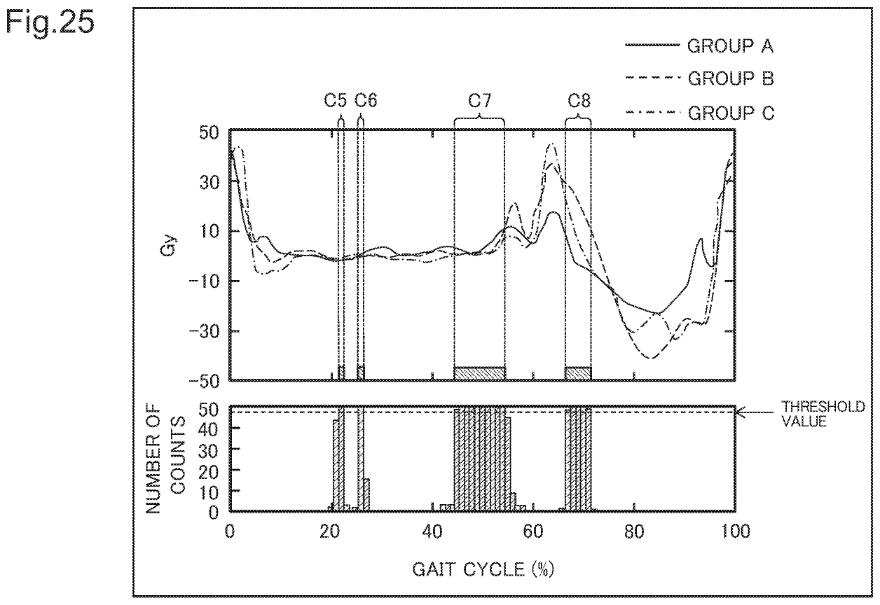

FIG. 25 illustrates an example in which the feature amount is extracted from the gait waveform of the angular velocity around the y-axis. The upper graph of FIG. 25 is a gait waveform Gy of the angular velocity around the y-axis. The gait waveform Gy is normalized so that 50 subjects can be compared under common conditions. In the example of FIG. 25, four feature amounts exceeding the threshold value of the count number are extracted among the plurality of extracted feature amounts. The first feature amount is a feature amount of a gait phase cluster C5 having a gait cycle of 22 to 23%. The gait phase cluster C5 is a feature amount extracted from a single gait phase, but is regarded as a cluster. The gait phase cluster C5 corresponds to the early stage of the mid-stance period. In the early stage of the mid-stance period, the sole is in contact with the ground. The second feature amount is a feature amount of a gait phase cluster C6 having a gait cycle of 27 to 28%. The gait phase cluster C6 is a feature amount extracted from a single gait phase, but is regarded as a cluster. The gait phase cluster C6 corresponds to the final stage of the mid-stance period. In the final stage of the mid-stance period leg, the sole is in contact with the ground. The third feature amount is a feature amount of a gait phase cluster C7 having a gait cycle of 46 to 56%. The gait phase cluster C7 corresponds to a period from the final stage of the mid-stance period to the pre-swing period. The fourth feature amount is a feature amount of a gait phase cluster C8 having a gait cycle of 68 to 72%. The gait phase cluster C8 corresponds to the final stage of the initial swing period.

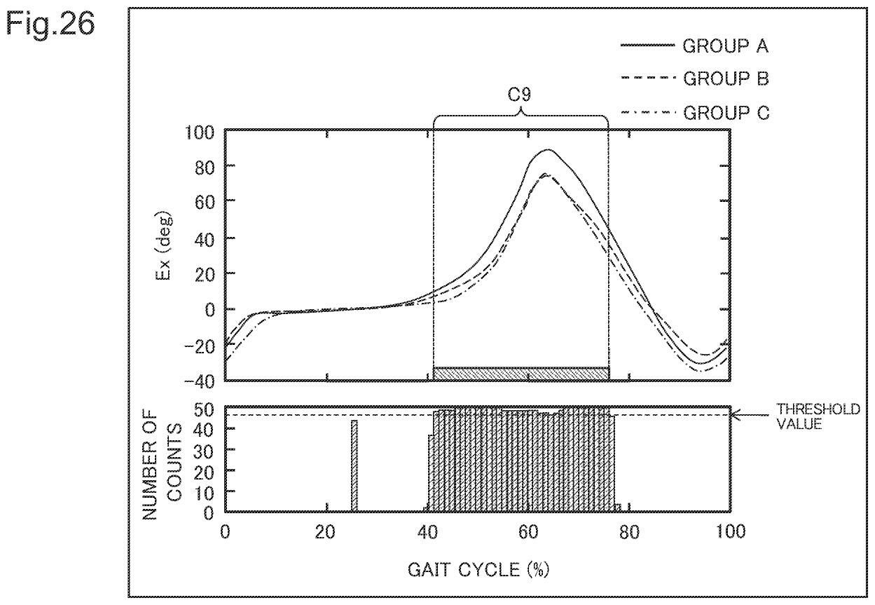

FIG. 26 illustrates an example in which the feature amount is extracted from the gait waveform of the plantar angle around the x-axis. The upper graph of FIG. 26 is a gait waveform Ex of the plantar angle about the x-axis. In the example of FIG. 26, one feature amount exceeding the threshold value of the count number is extracted among the plurality of extracted feature amounts. The extracted feature amount is a feature amount of a gait phase cluster C9 having a gait cycle of 41 to 77%. The gait phase cluster C9 corresponds to a period from the terminal stance period to the final stage of the mid-swing period.

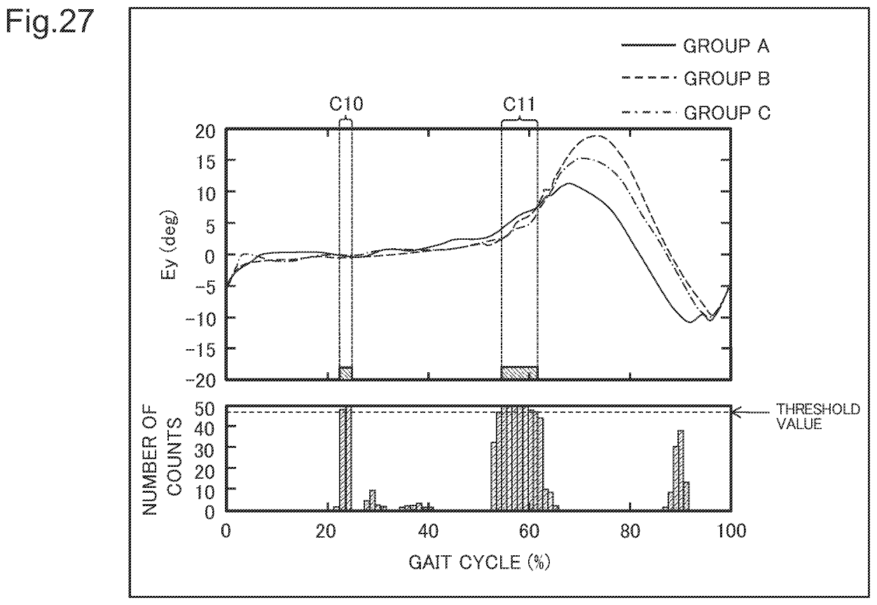

FIG. 27 illustrates an example in which the feature amount is extracted from the gait waveform of the plantar angle around the y-axis. The upper graph of FIG. 27 is a gait waveform Ey of the plantar angle about the y-axis. In the example of FIG. 27, two feature amounts exceeding the threshold value of the count number are extracted among the plurality of extracted feature amounts. The first feature amount is a feature amount of a gait phase cluster C10 having a gait cycle of 23 to 25%. The gait phase cluster C10 corresponds to the early stage of the mid-stance period. In the early stage of the mid-stance period, the sole is in contact with the ground. The second feature amount is a feature amount of a gait phase cluster C11 having a gait cycle of 54 to 63%. The gait phase cluster C11 corresponds to a period before and after the toe off.

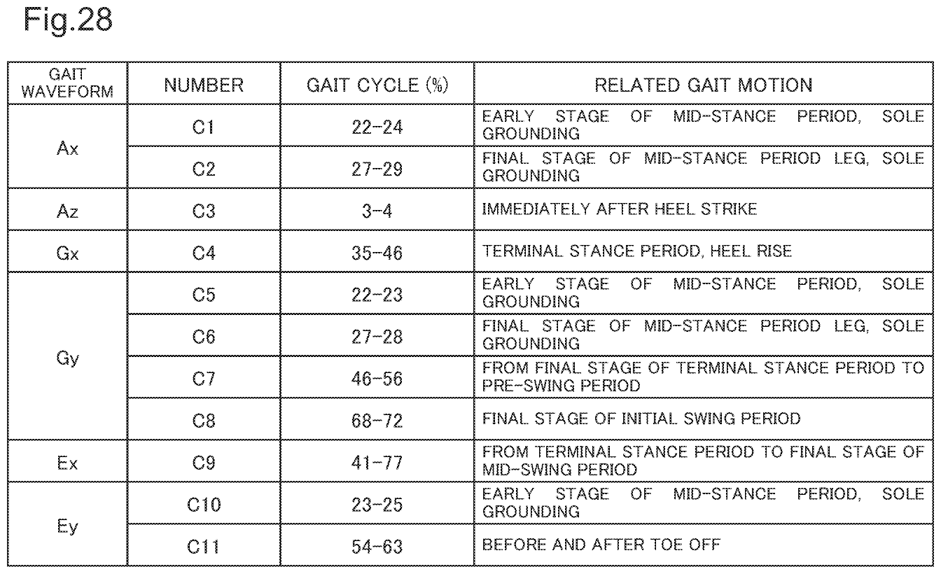

FIG. 28 is a correspondence table summarizing feature amounts extracted from the gait waveforms of FIGS. 22 to 27. The correspondence table of FIG. 28 associates the number of the gait phase cluster from which the feature amount is extracted, the gait cycle (%) from which the gait phase cluster is extracted, and the related gait motion for each gait waveform.

Next, a test example of an estimation model generated by trained with the feature-amount data generated based on the gait waveforms of FIGS. 22 to 27 will be described with an example. A test example of an estimation model that outputs an estimation value of the FMTPA in accordance with an input of feature-amount data will be described. In this test, the relationship between the estimation value of the estimation model generated by training using some algorithms and the true value was verified.

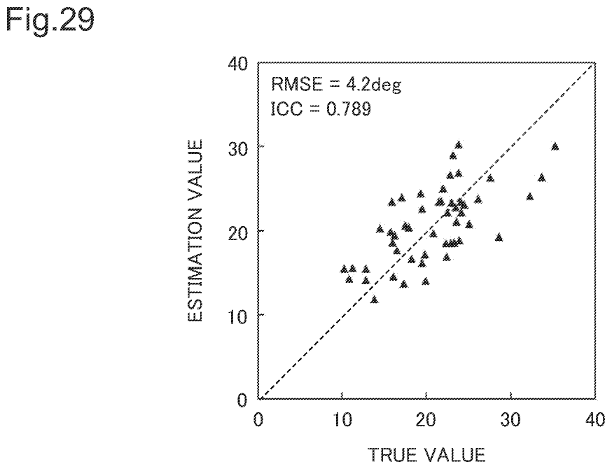

FIG. 29 is an example of the result of the cross test between the estimation result of the estimation model and the true value. FIG. 29 illustrates a relationship between an estimation result using an estimation model trained by linear regression and a true value. When an estimation model trained by linear regression is used, the root mean square error (RMSE) is 4.2 degrees, and the intra-class correlation coefficient (ICC) is 0.789. That is, it has been confirmed that the estimation model trained by linear regression outputs a good estimation value. By using this estimation model, the degree of progress of hallux valgus can be estimated with high accuracy with only 11 feature amounts.

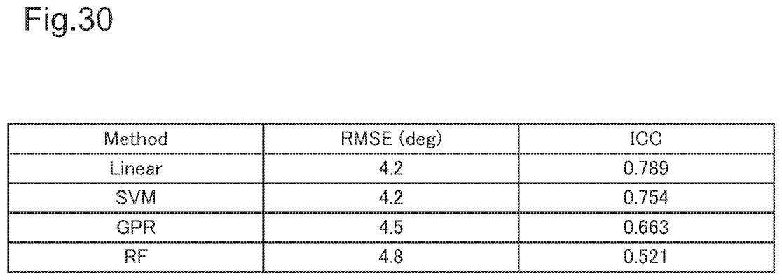

FIG. 30 is a table summarizing a relationship between an estimation value and a true value estimated using an estimation model generated by a plurality of algorithms. The table of FIG. 30 includes an RMSE and an ICC related to an estimation value using an estimation model trained by an algorithm such as linear regression (Linear), support vector machine (SVM), and Gaussian process regression (GPR), and random forest (RF). According to the table of FIG. 30, linear regression (Linear) had the smallest RMSE and the largest ICC. That is, in the example of FIG. 30, the estimation model trained by linear regression (Linear) has the best performance.

For example, a gait phase cluster from which a feature amount correlated with hallux valgus is extracted may be set in advance, and the feature amount of each of the gait phases (gait cycle) constituting the set gait phase cluster may be to be extracted. In other words, a gait phase cluster from which a feature amount correlated with the physical feature to be estimated is extracted may be set in advance, and the feature amount of each of the gait phases (gait cycle) constituting the set gait phase cluster may be to be extracted. In this way, when the feature amount to be extracted is set in advance according to the physical feature to be estimated, the data amount of the feature amount used for estimation can be reduced.

(Hardware)

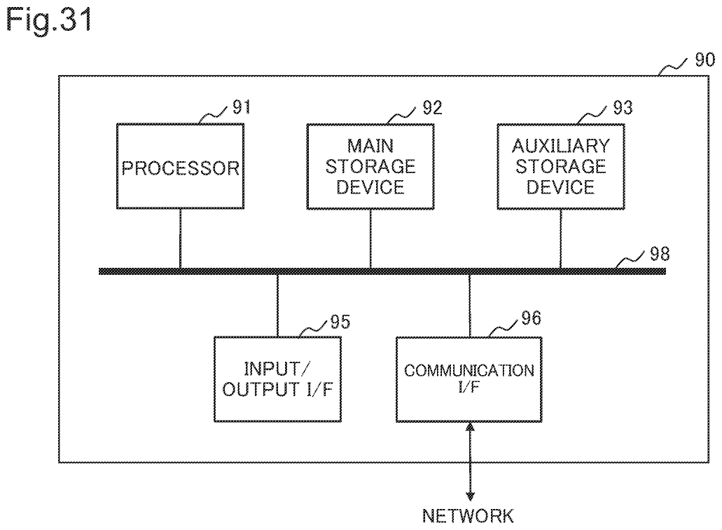

A hardware configuration for performing control and processing according to each example embodiment of the present disclosure will be described using an information processing device 90 of FIG. 31 as an example. The information processing device 90 in FIG. 31 is a configuration example for performing the process of the detection device of each example embodiment, and does not limit the scope of the present disclosure.

As illustrated in FIG. 31, the information processing device 90 includes a processor 91, a main storage device 92, an auxiliary storage device 93, an input/output interface 95, and a communication interface 96. In FIG. 31 the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, a software program installed in the information processing device 90 may be used. The processor 91 executes control and processing according to the present example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magnetoresistive random access memory (MRAM) may be configured and added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing device 90 with a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input of information and settings. In a case where the touch panel is used as the input device, the display screen of the display device may also serve as the interface of the input device. Data communication between the processor 91 and the input device may be mediated by the input/output interface 95.

The information processing device 90 may be provided with a display device that displays information. In a case where a display device is provided, the information processing device 90 preferably includes a display control device (not illustrated) that controls display of the display device. The display device may be connected to the information processing device 90 via the input/output interface 95.

The information processing device 90 may be provided with a drive device. The drive device mediates reading of data and a program from the recording medium, writing of a processing result of the information processing device 90 to the recording medium, and the like between the processor 91 and the recording medium (program recording medium). The drive device may be connected to the information processing device 90 via the input/output interface 95.

The above is an example of a hardware configuration for enabling control and processing according to each example embodiment of the present invention. The hardware configuration of FIG. 31 is an example of a hardware configuration for executing control and processing according to each example embodiment, and does not limit the scope of the present invention. A program for causing a computer to execute control and processing according to each example embodiment is also included in the scope of the present invention. A program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium is a program recording medium.

The components such as the feature-amount generation device, the data processing device, and the feature-amount generation device of each example embodiment may be combined in any manner. The components such as the feature-amount generation device, the data processing device, and the feature-amount generation device of each example embodiment may be achieved by software or may be achieved by a circuit.

While the present invention is described with reference to example embodiments thereof, the present invention is not limited to these example embodiments. Various modifications that can be understood by those of ordinary skill in the art can be made to the configuration and details of the present invention within the scope of the present invention.

### REFERENCE SIGNS LIST

10, 20, 40 gait measurement system
11, 21, 31, 41 measurement device
15, 25, 45 data processing device
30 training system
35 training device
110, 210 data acquisition unit
111, 211 acceleration sensor
112, 212 angular velocity sensor
113, 213 control unit
115, 255 feature-amount generation unit
116, 516 extraction unit
117, 517 generation unit
119, 219 transmission unit
151, 251, 351, 451 reception unit
157, 257 processing unit
353 training unit
355, 455 storage unit
457 estimation unit
460 mobile terminal
515 feature-amount generation device

1. A gait measurement system comprising:
a memory storing instructions, and
a processor connected to the memory and configured to execute the instructions to:
generate a gait waveform for one gait cycle from time-series data of sensor data measured according to a gait of a user;
extract a feature amount from the generated gait waveform;
extract a gait phase cluster by integrating a plurality of temporally continuous gait phases from each of which a feature amount is extracted;
generate a feature amount of the gait phase cluster using a preset feature-amount constitutive expression; and
estimate a degree of progress of hallux valgus of the user using the feature amount accumulated along with the gait of the user; and
display information related to the gait according to the degree of progress of hallux valgus of the user on a screen of a mobile terminal used by the user.

2. The gait measurement system according to claim 1, wherein
in a case where a feature amount is extracted from a single gait phase that is not temporally continuous,
the processor is configured to execute the instructions to
generate feature-amount data in which the single gait phase and the feature amount of the single gait phase are associated with each other.

3. The gait measurement system according to claim 1, wherein
in a case where a feature amount is extracted from a single gait phase that is not temporally continuous,
the processor is configured to execute the instructions to
extract the single gait phase as a gait phase cluster, and
generate feature-amount data in which the single gait phase extracted as the gait phase cluster and the feature amount of the single gait phase are associated with each other.

4. The gait measurement system according to claim 1, wherein
the processor is configured to execute the instructions to
extract the feature amount of the gait phases forming the walking phase cluster related to hallux valgus of the user based on preset conditions.

5. The gait measurement system according to claim 1, wherein
the processor is configured to execute the instructions to
extract a feature amount related to a gait affected by influence of hallux valgus.

6. The gait measurement system according to claim 1, wherein
the processor is configured to execute the instructions to
input the feature amount of the gait phase cluster extracted from the time-series data of the sensor data measured along with the gait of the user to a machine learning model that outputs the degree of progress of hallux valgus according to input the feature amount, and
estimate the degree of progress of hallux valgus of the user based on an estimation value output from the machine learning model.

7. The gait measurement system according to claim 6, wherein
the machine learning model is constructed by machine learning, and
the information is used for decision making to address the harmonic index.

8. The gait measurement system according to claim 1, further comprising
a data acquisition device configured to measure a spatial acceleration and a spatial angular velocity, and generate the sensor data based on the spatial acceleration and the spatial angular velocity.

9. An estimation method executed by a computer, the method comprising:
generating a gait waveform for one gait cycle from time-series data of sensor data measured according to a gait of a user;
extracting a feature amount from the generated gait waveform;
extracting a gait phase cluster by integrating a plurality of temporally continuous gait phases from each of which a feature amount is extracted;
generating a feature amount of the gait phase cluster using a preset feature-amount constitutive expression; and
estimating a degree of progress of hallux valgus of the user using the feature amount accumulated along with the gait of the user; and
displaying information related to the gait according to the degree of progress of hallux valgus of the user on a screen of a mobile terminal used by the user.

10. A non-transitory program recording medium recorded with a program causing a computer to perform the following processes:
generating a gait waveform for one gait cycle from time-series data of sensor data measured according to a gait of a user;
extracting a feature amount from the generated gait waveform;
extracting a gait phase cluster by integrating a plurality of temporally continuous gait phases from each of which a feature amount is extracted;
generating a feature amount of the gait phase cluster using a preset feature-amount constitutive expression; and
estimating a degree of progress of hallux valgus of the user using the feature amount accumulated along with the gait of the user; and
displaying information related to the gait according to the degree of progress of hallux valgus of the user on a screen of a mobile terminal used by the user.
